(19) United States
(12) Patent Application Publication
(10) Pub. No.: US 2025/0270576 A1
(43) Pub. Date: Aug. 28, 2025

(54) AGRICULTURAL COMPOSITIONS AND METHODS OF USING THE SAME

(71) Applicant: Arizona Board of Regents on behalf of Arizona State University, Scottsdale, AZ (US)

(72) Inventors: Ferran GARCIA-PICHEL, Phoenix, AZ (US); Roberto GAXIOLA, Tempe, AZ (US)

(21) Appl. No.: 18/576,311

(22) PCT Filed: Jul. 5, 2022
(86) PCT No.: PCT/US2022/036109

§ 371 Date: Jan. 3, 2024

Related U.S. Application Data
(60) Provisional application No. 63/218,470, filed on Jul. 5, 2021.

(51) Int. Cl. C12N 15/82; A01N 31/02; A01N 63/20; A01P 21/00; C12N 9/14

(52) U.S. Cl. CPC C12N 15/8261; A01N 31/02; A01N 63/20; A01P 21/00; C12N 9/14; C12N 15/8273

(57) ABSTRACT

This invention is directed agricultural compositions and methods of using the same. For example, this invention is directed to agricultural compositions comprising an enriched root associated microbiome, and methods of using the same to increase plant growth or plant yield, or improving soil quality.

This application is a National Stage Entry of PCT Application No. PCT/US2022/036109, filed on Jul. 5, 2022, which is an International Application that claims priority from U.S. Provisional Application Ser. No. 63/218,470 filed on Jul. 5, 2021, the contents of each of which are incorporated herein by reference in their entireties.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed agricultural compositions and methods of using the same. For example, this invention is directed to agricultural compositions comprising an enriched root associated microbiome, and methods of using the same to increase plant growth or plant yield, or improving soil quality.

### BACKGROUND OF THE INVENTION

Genetic modification of crop genotypes to provide for phenotypes is standard in agriculture, based on the correspondence between genotype and observed phenotype. For example, the expression of an organism's genes determines its own characteristics in a given environment.

### SUMMARY OF THE INVENTION

An aspect of the invention is directed to methods of increasing plant growth or plant yield.

In embodiments, the method comprises co-cultivating a first plant and a second plant, wherein the first plant comprises a genetically engineered plant that produces an enriched root associated microbiome (RAM), wherein the enriched RAM increases plant growth and/or plant yield of the second plant.

Aspects of the invention are also directed to methods of improving soil quality.

In embodiments, the method comprises cultivating in soil a genetically engineered plant that produces an enriched root associated microbiome (RAM), wherein the enriched RAM improves the soil quality.

In embodiments, increased plant growth, increased plant yield, or improved soil quality can be indicated by increased root yield, increased shoot yield, or increased crop yield. For example, plant yield can comprise plant foliage and/or crop yield.

In embodiments, the genetically engineered plant can be engineered to over express a type 1 H+-pyrophosphatase.

In embodiments, the genetically engineered plant can be of the genus Arabidopsis. For example, the Arabidopsis plant can be Arabidopsis thaliana.

In embodiments, the second plant can be a wildtype plant.

In embodiments, the second plant can be a crop plant. For example, the crop plant can be selected from the group consisting of rice, barley, tobacco, cotton, alfalfa, maize, and wheat.

In embodiments, the enriched RAM comprises one or more viable microorganisms. For example, the one or more viable microorganisms can be selected from the group consisting of bacteria, fungi, yeast, unicellular algae, viruses, and phages. For example, the one or more viable microorganisms can be selected from the group consisting of Proteobacteria, Actinobacteria, Verrucomicrobia, Chloroflexi, Nitrospirae, Bacteriodetes, Acidobacteria, Planctomycetes, Gemmatimonadetes, Firmicutes, or any combination thereof.

In embodiments, the one or more viable microorganisms can comprise enriched fermentation capacity. For example, the enriched fermentation capacity comprises butanediol fermentation.

Aspects of the invention are also drawn to methods of increasing plant growth or plant yield.

In embodiments, the method comprises applying to the soil of a plant an agricultural composition comprising an enriched root associated microbiome (RAM) or a portion thereof.

Still further, aspects of the invention are drawn to a method of improving soil quality.

In embodiments, the method comprises applying to soil an agricultural composition comprising an enriched root associated microbiome (RAM) or a portion thereof.

In embodiments, the agricultural composition comprising an enriched root associated microbiome (RAM) can be provided in an amount effective to increase plant growth, increase plant root yield, increase leaf foliage, increase plant shoot yield, increase crop yield, and/or improve soil quality.

In embodiments, the enriched RAM comprises one or more viable microorganisms. For example, the one or more viable microorganisms can be selected from the group consisting of bacteria, fungi, yeast, unicellular algae, viruses, and phages. For example, the one or more viable microorganisms can be selected from the group consisting of Proteobacteria, Actinobacteria, Verrucomicrobia, Chloroflexi, Nitrospirae, Bacteriodetes, Acidobacteria, Planctomycetes, Gemmatimonadetes, Firmicutes, or any combination thereof.

In embodiments, the one or more viable microorganisms can comprise enriched fermentation capacity. For example, the enriched fermentation capacity comprises butanediol fermentation.

In embodiments, the enriched root associated microbiome (RAM) can be obtained by cultivating a genetically engineered plant in a soil composition for a period of time, thereby providing a composition comprising an enriched RAM, and obtaining therefrom the composition comprising the enriched RAM.

Aspects of the invention are also drawn to an agricultural composition.

In embodiments, the agricultural composition comprises an enriched root associated microbiome (RAM) or a portion thereof, wherein the enriched RAM is obtained from a genetically engineered plant.

In embodiments, the genetically engineered plant can be modified to overexpress a type 1 H+-pyrophosphatase.

In embodiments, the genetically engineered plant can be of the genus Arabidopsis. For example, the Arabidopsis plant can be Arabidopsis thaliana.

In embodiments, the enriched RAM comprises one or more viable microorganisms. For example, the one or more viable microorganisms can be selected from the group consisting of bacteria, fungi, yeast, unicellular algae, viruses, and phages. For example, the one or more viable microorganisms can be selected from the group consisting of Proteobacteria, Actinobacteria, Verrucomicrobia, Chloroflexi, Nitrospirae, Bacteriodetes, Acidobacteria, Planctomycetes, Gemmatimonadetes, Firmicutes, or any combination thereof.

In embodiments, the one or more viable microorganisms can comprise enriched fermentation capacity. For example, the enriched fermentation capacity comprises butanediol fermentation.

In embodiments, the enriched RAM can further comprise metabolites of nitrogen metabolism.

In embodiments, the enriched RAM can further comprise 2,3 butanediol.

In embodiments, the agricultural composition can further comprise at least one plant nutrient.

Still further, aspects of the invention are drawn towards an agricultural composition comprising an enriched root associated microbiome (RAM).

In embodiments, the agricultural composition can be produced by cultivating a genetically engineered plant in a soil composition such that the genetically engineered plant produces an enriched RAM within the soil composition, thereby providing the agricultural composition comprising an enriched RAM.

In embodiments, the genetically engineered plant can be removed from the agricultural composition once the enriched RAM is produced.

In embodiments, the genetically engineered plant can be of the genus Arabidopsis. For example, the Arabidopsis plant can be Arabidopsis thaliana.

In embodiments, the enriched RAM comprises one or more viable microorganisms. For example, the one or more viable microorganisms can be selected from the group consisting of bacteria, fungi, yeast, unicellular algae, viruses, and phages. For example, the one or more viable microorganisms can be selected from the group consisting of Proteobacteria, Actinobacteria, Verrucomicrobia, Chloroflexi, Nitrospirae, Bacteriodetes, Acidobacteria, Planctomycetes, Gemmatimonadetes, Firmicutes, or any combination thereof.

In embodiments, the one or more viable microorganisms can comprise enriched fermentation capacity. For example, the enriched fermentation capacity comprises butanediol fermentation.

In embodiments, the enriched RAM can further comprise metabolites of nitrogen metabolism.

In embodiments, the enriched RAM can further comprise 2,3 butanediol.

In embodiments, the agricultural composition can further comprise at least one plant nutrient.

Aspects of the invention are also drawn towards a method of manufacturing an agricultural composition. In embodiments, the agricultural composition can comprise an enriched root associated microbiome (RAM).

In embodiments, the method comprises cultivating a genetically engineered plant in a soil composition such that the genetically engineered plant produces an enriched RAM within the soil composition, thereby providing an agricultural composition comprising an enriched RAM.

Further, aspects of the invention are drawn towards a method of manufacturing a soil-specific agricultural composition.

In embodiments, the method comprises obtaining a soil sample and cultivating a genetically engineered plant in the soil sample such that the genetically engineered plant produces an enriched RAM within the soil sample, thereby providing a soil-specific agricultural composition comprising an enriched RAM.

In embodiments, the genetically engineered plant can be removed from the agricultural composition once the enriched RAM is produced.

In embodiments, the genetically engineered plant can be of the genus Arabidopsis. For example, the Arabidopsis plant Arabidopsis thaliana.

In embodiments, the enriched RAM comprises one or more viable microorganisms.

For example, the one or more viable microorganisms can be selected from the group consisting of bacteria, fungi, yeast, unicellular algae, viruses, and phages. For example, the one or more viable microorganisms can be selected from the group consisting of Proteobacteria, Actinobacteria, Verrucomicrobia, Chloroflexi, Nitrospirae, Bacteriodetes, Acidobacteria, Planctomycetes, Gemmatimonadetes, Firmicutes, or any combination thereof.

In embodiments, the one or more viable microorganisms can comprise enriched fermentation capacity. For example, the enriched fermentation capacity comprises butanediol fermentation.

Other objects and advantages of this invention will become readily apparent from the ensuing description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows phenotypic transfer between wild type A. thaliana (Col-0) and its derived transgenic AVP1. Photographs show phenotypic aspects of shoots (top row) as well as shoots and roots (second row) of plants grown as neighbor pairs on artificial soil. Genotypes indicated in top row and apply to both rows. Bottom panel: root and shoot yield of the two genotypes grown as homo- or heterogenetic pairs on different soils, with 16>n>14 per genotype. Error bars are +/−s.d.

FIG. 2 shows extension of the plant genotype to the root-associated microbiome and its transferability. Upper left panel: microbial biomass and diversity in three different soils (n=9 plants pooled into 3 determinations). Upper right-hand panel: 16S rRNA gene-based microbiome composition through principal component analyses (ellipses denote 95% CI; n=9 plants pooled into 3 determinations). Bottom left panel: traits of rhizosphere microbiomes from plants grown as homogenetic or heterogenetic pairs on artificial soils (n=18 plants pooled into 6 determinations). Lower right-hand panel: plant phenotypes grown under sterility.

FIG. 3 shows mechanisms for phenotype extension to the root-associated microbiomes and for their transferability. Top left: phenotypes of Col-0 (wild type; Arabidopsis thaliana) and genetically modified plants expressing the transgenic PROTON (H+-) pyrophosphatase in all tissues (AVP1) or in phloem cells only (pCoY), where average yields are AVP1>pCoY>Col-0 (FIG. 7). Left center: differential root acidification capacity of each genotype. Left bottom: 16S rRNA based composition of the root associated microbiomes (RAMs) through principal component analyses. Middle: Metabolomic profile of root exudates of the three plant genotypes, depicted as a heat map of relative concentration (each column a pools n>3 plants). Top right: Enrichment of proportional abundance of query and control pathways in the metagenome of rhizosphere RAMs. Middle right: Effect of exposure to 250 μM 2,3-butanediol on root development of Col-0 plants. Bottom right: mechanistic model for plant-microbe interactions at the base of phenotype enhancement and transferability.

FIG. 4 shows compositional differences among RAMs in soils used for experimentation, based on 16S rRNA sequences, including determinations on rhizosphere RAM, and BRAM, for Col-0 and AVP1 plants. Differences among soils are much larger than those between plants in the same soils. AS: artificial soil. AZ: Arizona soil. IA: Iowa soil.

FIG. 5 shows RAM community composition at the Phylum level, based on 16S rRNA sequences and bioinformatic placement, for different RAM fractions, plant genotypes, genotypic neighbor, or soil substrates, as indicated in the left axis. Absolute values are in the bar graphs to the left, and relative composition to the right. Each condition has 3 composite samples independently processed with respect to incubation, sampling and analyses.

FIG. 6 shows volcano plots of relative abundance for individual 16S IRNA gene bacterial sequences between the rhizosphere RAMS of AVP1 and Col-0, when grown on different substrates. Sequences the differential abundance of was significantly different are denoted by red dots.

FIG. 7 shows biomass yields for wild type (Col-0) A. thaliana plants and the derived proton pyrophosphate transgenics pCOY and AVP1, relative to the yield of the wild type.

FIG. 8 shows key for accessing metabolomics LCMS raw and processed data as a zip file after creating a user account from https://genome.jgi.doe.gov/portal/201Tratabolomics_FD/201Tratabolomics_FD.info.html under data ID numbers 1266727 and AP, respectively.

FIG. 9 is a schematic showing the design of an experiment comparing the growth of a wild type plant when the soil is inoculated with the microbiome of from a wild type plant or a genetically engineered plant.

FIG. 10 shows the experimental results of an experiment comparing the growth of a wild type plant when the soil is inoculated with the microbiome of from a wild type plant or a genetically engineered plant.

FIG. 11 shows that inoculation of soil with the microbiome from a genetically engineered plant improves the wild-type below-ground performance.

FIG. 12 shows that inoculation of soil with the microbiome from a genetically engineered plant improves the wild-type above-ground performance.

### DETAILED DESCRIPTION OF THE INVENTION

The expression of an organism's genes determines its own characteristics in a given environment. And yet, surprisingly, we found that the phenotype of transgenic plants genetically modified to enhance yield transfers readily to neighboring wild type plants. We show that the transgenic plant's phenotype extends to its root-associated microbiome (RAM), which is larger, of different specific composition and functional activity than that of the wild type plant, as a result of altered root exudation. We provide evidence that the RAM of the transgenic plant mediates its transfer to neighboring plants through the production of, for example, fermentative 2,3-butanediol.

### Abbreviations and Definitions

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Aspects of the invention comprise a method of increasing plant growth or plant yield.

The term “plant growth” can refer to the growth of any plant part, including stems, leaves and roots. Growth can refer to the rate of growth, size, or number of any plant or plant part, including the plant shoot or plant root. In embodiments, plant growth can be increased by about 10%, but about 20%, by about 30%, by about 40%, by about 50%, by about 60%, by about 70%, by about 80%, by about 90%, by about 100%, or greater than 100%. In embodiments, plant growth can be increased relative to a control plant.

The term “plant yield” can refer to the amount of produced biological material, and in embodiments, can be used interchangeably with “biomass”. For crop plants, “yield” can also mean the amount of harvested material per unit of production or per area (e.g. hectare). Yield can be defined in terms of quantity or quality. The harvested material can vary from crop to crop, for example, it can be seeds, above-ground biomass, below-ground biomass (e.g. potatoes), roots, fruits, or any other part of the plant which is of economic value, including foliage or flowers. “Yield” can also encompass yield stability of the plants. “Yield” can also encompass yield potential, which is the maximum obtainable yield under optimal growth conditions. Yield can be dependent on a number of yield components, which can be monitored by certain parameters. These parameters are well known to persons skilled in the art and vary from crop to crop. For example, breeders are well aware of the specific yield components and the corresponding parameters for the crop they are aiming to improve. For example, key yield parameters for potato include tuber weight, number of tubers, and number of stems per plant.

For example, plant yield can be increased by about 10%, but about 20%, by about 30%, by about 40%, by about 50%, by about 60%, by about 70%, by about 80%, by about 90%, by about 100%, or greater than 100%. In embodiments, plant yield can be increased relative to a control plant.

Aspects of the invention can also be drawn to increasing plant productivity. The term “plant productivity” can refer to any aspect of growth or development of a plant, including those aspects for which the plant is grown. For example, improved or increased plant productivity can refer to improvements in biomass or yield of any part of a plant, including leaves, stems, roots, grain, fruit, vegetables, flowers, or other plant parts harvested or used for various purposes, and improvements in growth of plant parts, including stems, leaves and roots. For example, when referring to food crops, such as grains, fruits or vegetables, plant productivity can refer to the yield of grain, fruit, vegetables or seeds harvested from a crop. For crops such as pasture, plant productivity can refer to growth rate, plant density or the extent of groundcover.

For example, plant productivity can be increased by about 10%, but about 20%, by about 30%, by about 40%, by about 50%, by about 60%, by about 70%, by about 80%, by about 90%, by about 100%, or greater than 100%. In embodiments, plant yield can be increased relative to a control plant.

In embodiments, methods described herein can comprise co-cultivating a first plant (for example, a genetically engineered plant) and a second plant (for example, a wild type plant). For example, the first plant and the second plant can be co-cultivated such that the phenotype of the genetically engineered first plant is transferred readily to the neighboring wild type plant via an enriched root associated microbiome produced by the first plant.

In embodiments, the term “cultivating” can refer to the process of growing a plant or a crop in a medium, such as a soil composition.

In embodiments, the term “co-cultivating” can refer to the simultaneous cultivation of two or more species of a plant in the same medium, such as in the same soil composition.

In embodiments, the two or more species of a plant can be co-cultivated less than about 1 cm apart (at the shoot level or at the root level), about 1-2 cm apart, about 2-3 cm apart, about 3-4 cm apart, about 5-cm cm apart, about 1-5 cm apart, about 5-10 cm apart, about 10-25 cm apart, about 25-50 cm apart, about 50-75 cm apart, about 75-100 cm apart, about 1-2 meters apart, about 2-5 meters apart, about 5-10 meters apart, or greater than 10 meters apart. The skilled artisan will recognize that the distance between the two or more species depends on various factors, including the type of plant, the type of soil, and/or the growth conditions. In embodiments, the two or more species of plants are co-cultivated by a distance that is sufficient to allow the roots of both plants to interact.

In embodiments, the term “co-cultivating” can refer to the sequential cultivation of two or more species of a plant in the same medium, such as in the same soil composition. For example, a first plant can be cultivated in a soil composition for a period of time, thereby providing or producing an enriched root associated microbiome (RAM). Optionally, the first plant can be removed from the soil composition (comprising the enriched root associated microbiome), and one or more second plants can be planted in the soil composition comprising the enriched RAM, thereby increasing plant growth and/or plant yield of the second plant.

In embodiments, the first plant can provide or produce an enriched root associated microbiome (RAM), wherein the enriched RAM increases plant growth and/or plant yield of the second plant.

In embodiment, the first plant can be a genetically engineered plant. A “genetically engineered plant” can refer to a plant that has been genetically manipulated. Genetic manipulation can include recombinant DNA engineering as well as other forms of altering the amount, nature, or activity of nucleic acids in a plant, such as mutagenizing plant by exposing it to a mutagen such as UV light. The term “transgenic” is used in its broadest sense and can refer to an organism wherein at least one exogenous nucleotide sequence has been introduced into the cell. An “exogenous” nucleotide sequence can refer to a nucleotide sequence that has been introduced into a cell (or an ancestor of a cell) using genetic engineering techniques. An exogenous nucleotide sequence can be introduced into a plant cell using a vector. An exogenous nucleic acid can include a nucleic acid that is foreign, i.e., heterologous with respect to the host cell's genome; but an exogenous nucleotide sequence can also encode an enzyme that is endogenous to the cell into which it is introduced. For example, exogenous nucleic acids include those nucleic acids designed to overproduce endogenous enzymes, such as a type of H+-pyrophosphatase. The exogenous nucleic acid(s) in the genetically engineered plant of the invention can be stable and inheritable. The exogenous nucleic acids can integrate into the plant genome.

In embodiments, genetically engineered plants can be characterized by increased or elevated levels of a type 1 H+-pyrophosphatase. See, for example, Genbank Accession No. At1 g15690. The gene for type I H⁺-pyrophosphatases are a yield enhancement determinant whose overexpression triggers production of more, larger leaves and root biomass in genetically engineered Arabidopsis thaliana over the wild type. These mutants also possess increased salinity and drought resistance. Similar differential phenotypes of H⁺-PPase overexpressing transgenics are seen in crop plants including rice (Oryza sativa), barley (Hordeum vulgare), tobacco (Nicotiana tabacum), lettuce (Lactuca sativa), peanuts (Arachis hypogaea), sweet potato (Ipomoea batatas) cotton (Gossypium hirsutum), alfalfa (Medicago sativa), maize (Zea mays), and wheat (Triticum aestivum). Another very conserved phenotype associated with these genetically modified plants is an increased acidification of the soil surrounding roots. Because these traits are governed by plant physiological mechanisms, we were surprised to find that the phenotype of a genetically engineered plant of the genus Arabidopsis (with ubiquitous expression of H⁺-PPase) can be transferred to nearby wild-type plants.

In addition to Arabidopsis thaliana, the genetically engineered plant as described herein can be any species of the genus Arabidopsis. For example, the Arabidopsis plant can be Arabidopsis Arenicola, Arabidopsis arenosa, Arabidopsis cebennensis, Arabidopsis croatica, Arabidopsis helleri, Arabidopsis lyrate, Arabidopsis neglecta, Arabidopsis pedemontana, Arabidopsis suecica, or Arabidopsis thaliana. In embodiments, the genetically engineered plant is Arabidopsis thaliana.

The genetically-engineered plant can be a crop plant, as described herein. The term “crop plant” or “crop” can refer to any plant grown to be harvested or used for any economic purpose, including for example human foods, livestock fodder, fuel or pharmaceutical production. The crop plant can be, for example, a food crop (for humans or other animals) such as any fruit, vegetable, nut, seed or grain producing plant. Exemplary crop plants include, but are not limited to, tubers and other below-ground vegetables (such as potatoes, beetroots, radishes, carrots, onions, etc.), ground-growing or vine vegetables (such as pumpkin and other members of the squash family, beans, peas, asparagus, etc.), leaf vegetables (such as lettuces, chard, spinach, alfalfa, etc.), other vegetables (such as tomatoes, brassica including broccoli, avocadoes, etc.), fruits (such as berries, olives, stone fruits including nectarines and peaches, tropical fruits including mangoes and bananas, apples, pears, mandarins, oranges, mandarins, kiwi fruit, coconut, etc.), cereals (such as rice, maize, wheat, barley, millet, oats, rye etc.), nuts (such as macadamia nuts, peanuts, brazil nuts, hazel nuts, walnuts, almonds, etc.), and other economically valuable crops and plants (such as corn, sugar cane, soybeans, sunflower, canola, sorghum, pastures, turf grass, etc). Non-limiting examples of crop plants include rice, barley, tobacco, cotton, alfalfa, maize, and wheat.

In certain embodiments, the first plant is a natural plant that has been selectively bred to acquire specific characteristics (for example, natural plant variants). For example, the natural plant variant has increased H⁺-PPase expression. For example, the term “natural plant” can encompass plants that have not been genetically modified (i.e., transgenic). Non-limiting examples comprise certain lines of corn, for example, drought tolerant allele ZmVPP1 from two drought-tolerant inbred lines (CIMBL70 and CIMBL91). See, for example, Wang X, Wang H, Liu S, Ferjani A, Li J, Yan J, Yang X, Qin F (2016) Genetic variation in ZmVPP1 contributes to drought tolerance in maize seedlings. Nature Genetics: 1-12

In embodiments, the second plant can be a wild type plant. The term “wild type”, for example, can refer to the corresponding not genetically modified starting organism. For example, the term “wild type plant” can refer to the plant or plant species used as starting material for the manufacture of a genetically modified plant. In embodiments, the second plant does not have increased H+-PPase expression. In embodiments, the term “wild type” can also refer to a reference organism.

In certain embodiments, the second plant can be a genetically engineered plant, but one which is modified differently than the first plant. For example, the second plant can be modified to be pesticide resistant, herbicide resistant, or draught tolerant. In embodiments, the second plant is not genetically modified to have increased H+-PPase expression.

In embodiments, the second plant comprises a crop plant. The term “crop plant” or “crop” can refer to any plant grown to be harvested or used for any economic purpose, including for example human foods, livestock fodder, fuel or pharmaceutical production. The crop plant can be, for example, a food crop (for humans or other animals) such as any fruit, vegetable, nut, seed or grain producing plant. Exemplary crop plants include, but are not limited to, tubers and other below-ground vegetables (such as potatoes, beetroots, radishes, carrots, onions, etc.), ground-growing or vine vegetables (such as pumpkin and other members of the squash family, beans, peas, asparagus, etc.), leaf vegetables (such as lettuces, chard, spinach, alfalfa, etc.), other vegetables (such as tomatoes, brassica including broccoli, avocadoes, etc.), fruits (such as berries, olives, stone fruits including nectarines and peaches, tropical fruits including mangoes and bananas, apples, pears, mandarins, oranges, mandarins, kiwi fruit, coconut, etc.), cereals (such as rice, maize, wheat, barley, millet, oats, rye etc.), nuts (such as macadamia nuts, peanuts, brazil nuts, hazel nuts, walnuts, almonds, etc.), and other economically valuable crops and plants (such as corn, sugar cane, soybeans, sunflower, canola, sorghum, pastures, turf grass, etc). Non-limiting examples of crop plants include rice, barley, tobacco, cotton, alfalfa, maize, and wheat.

In embodiments, the first plant (i.e., the genetically engineered plant) can be co-cultivated with one or more plants, such as 2 plants, 3 plants, 4 plants, 5 plants, 6 plants, 7 plants, 8 plants, 9 plants, 10 plants, 25 plants, 100 plants, or more than 100 plants.

In embodiments, increased plant growth or increased plant yield can be indicated by increased root yield, increased shoot yield, or increased crop yield. The “root” of a plant can refer to the fibrous, underground part of a plant associated with mineral and water absorption. Functions of the root include anchorage of the plant, absorption of water and dissolved minerals and conduction of these to the stem, acidification od the soil solution, and storage of foods. The “root zone” can refer to a portion of the soil column occupied by plant roots. For example, increased root development can improve water and nutrient use efficiencies. For example, increased root development and acidification capacity can augment nutrient contents in the shoots, fruits and seeds (i.e., iron, zinc, potassium, nitrogen, phosphate).

In embodiments, root yield can increase by about 10%, about 20%, about 30%, about 40%, about 50%, about 60%, about 70%, about 80%, about 90% or about 100%. In embodiments, the root yield can refer to root biomass. For example, the root yield can refer to the root biomass of a plant with an edible root.

The “shoot” of a plant can refer to the plant stem, together with its appendages, leaves and lateral buds, flowering stems, and flower buds. The new growth from seed germination that grows upwards is a shoot where leaves will develop. Stems, which are an integral component of shoots, provide an axis for buds, fruits, and leaves. In embodiments, the shoot yield can refer to the shoot biomass. For example, the shoot yield can refer to the shoot biomass of a plant with an edible shoot.

In embodiments, shoot yield can increase by about 10%, about 20%, about 30%, about 40%, about 50%, about 60%, about 70%, about 80%, about 90% or about 100%.

The term “crop yield” can refer to the return of crop material obtained after harvesting a plant crop. In embodiments, crop yield can be root or shoot biomass or fruit production or seed production. An increase in crop yield can refer to an increase in crop yield relative to an untreated control crop. For example, in embodiments, yield, such as root yield, shoot yield, or crop yield, can be determined by comparing the root, shoot, and/or crop of a test plant with that of control plants grown, such as control plants grown with same watering and fertilization regimens but without the addition of a modified root microbiome.

In embodiments, crop yield can increase by about 10%, about 20%, about 30%, about 40%, about 50%, about 60%, about 70%, about 80%, about 90% or about 100%.

As described herein, the first plant can provide or produce an enriched “root associated microbiome (RAM)”, which is the dynamic assemblage of microorganisms living in the rhizosphere, such as 0-1 mm, 1-3 mm, 3-5 mm, 5-10 mm, 10-20 mm, 20-50 mm, 50-75 mm, 75-100 mm, 100-200 mm, 200-500 mm, 500-750 mm, or 750-1000 mm region adjacent to the external surface of the root. In embodiments, the RAM can refer to the dynamic assemblage of microorganisms living in the rhizosphere, such as 1-3 mm region adjacent to the external surface of the root. Because they are rich in a variety of carbon compounds, plant roots provide unique environments for a diverse assemblage of soil microorganisms, including bacteria, fungi and archaea.

In embodiments, the enriched RAM can comprise one or more viable microorganisms, one or more proteins, one or more sugars, one or more volatile organic compounds (VOCs), or any combination thereof. For example, the proteins, sugars, or VOCs can be released by the root of the plant, or by the microorganisms.

The term “protein” can refer to an amino acid polymer. The amino acids can be natural or non-natural amino acids D- and L-amino acids as is well understood in the art. The term “peptide” can refer to a protein with up to 50 amino acids. The term “polypeptide” can refer to a protein with more than 50 amino acids.

The term “sugar” can refer to monosaccharides, disaccharides, trisaccharides, oligosaccharides and polysaccharides. For example, the roots can secrete oligosaccharides and signaling molecules

The term “volatile organic compounds (VOCs)” can refer to a carbon containing compound that can be released from a composition, such as a plant or a microorganism. For example, the volatile organic compound is 2,3 butanediol (for example, its boiling point is about 177° C.) that can be produced by microorganisms in soil. 2,3 butanediol is a key product of bacterial fermentation, and can also act as a growth inducing hormone.

The term “microbial volatile organic compounds” can refer to a variety of compounds formed in the metabolism of fungi and bacteria.

The term “microorganism” can refer to a unicellular or multi-cellular microscopic or macroscopic life form. Microorganisms can include, but are not limited to, bacteria, protobacteria, protozoa, phytoplankton, viruses, fungi, yeast, phages, algae, oomycetes, parasites, nematodes, and any combination thereof.

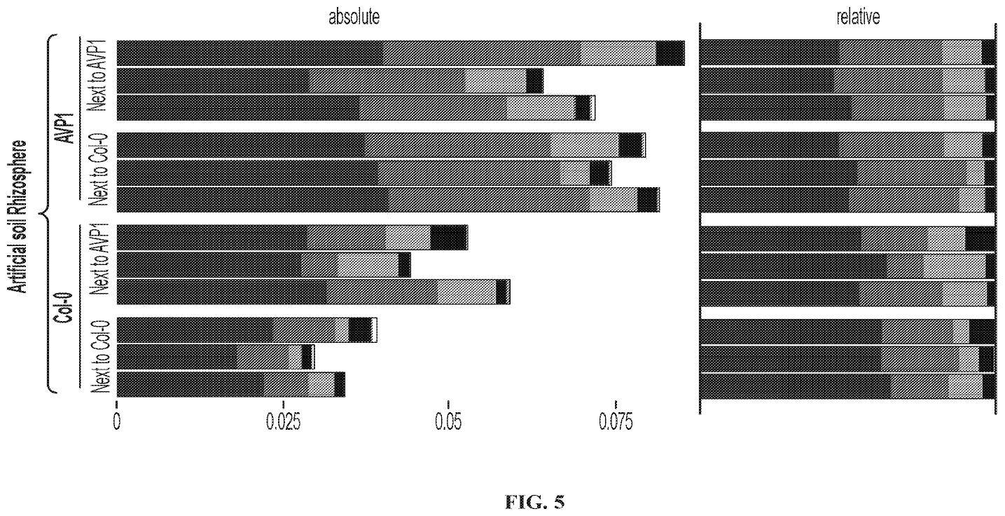

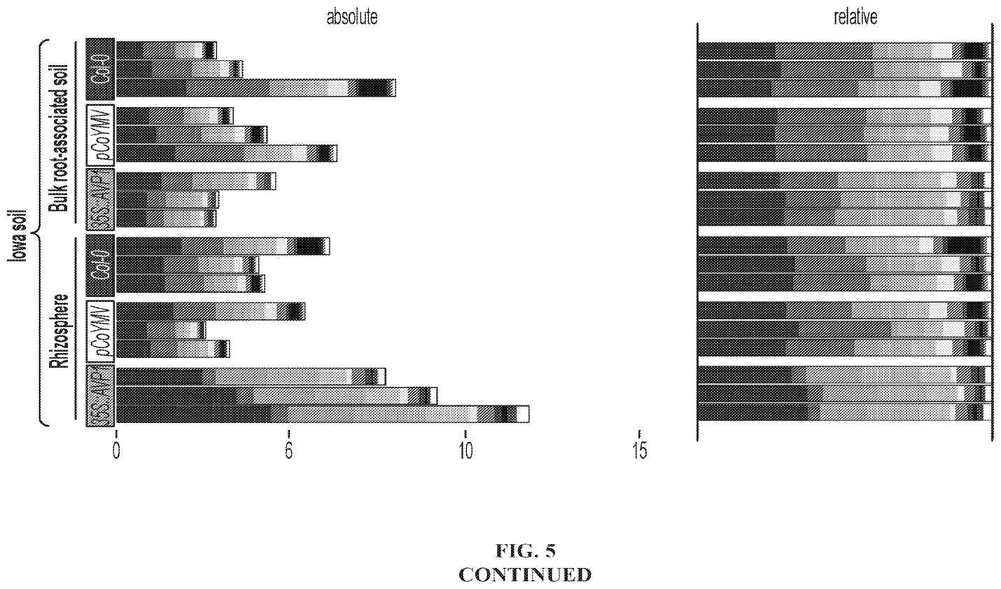

The term “viable microorganism” can refer to a microorganism that is substantially unaltered from its native state and comprises normal metabolic activity, including reproduction. Referring to FIG. 5, for example, the one or more viable microorganisms can comprise Proteobacteria, Actinobacteria, Verrucomicrobia, Chloroflexi, Nitrospirae, Bacteriodetes, Acidobacteria, Planctomycetes, Gemmatimonadetes, Firmicutes, or any combination thereof. In embodiments, the viable microorganism can be selected on the basis of containing specific metabolic capabilities.

In embodiments, the viable microorganisms can comprise enriched fermentation capacity. The term “fermentation” can refer to the enzymatic and anaerobic breakdown of organic substances by microorganisms to produce simpler organic compounds.

While fermentation occurs under anaerobic conditions it is not intended that the term be solely limited to strict anaerobic conditions, as fermentation also occurs in the presence of oxygen (e.g., under microaerophilic and other conditions).

The term “enriched fermentation capacity” can refer to a level of fermentation that is present at a relative or absolute level that is higher than a normal sample or a control sample. For example, the enriched fermentation capacity can comprise on capacity butanediol fermentation. For example, butanediol fermentation can refer to a type of fermentation in which organic compounds, such as sugars, are fermented with the production of 2,3-butanediol and other substances.

In embodiments, the enriched fermentation capacity can be gauged as the abundance of the sum of fermentation genes in the RAM. Non-limiting examples of such fermentation genes include those shown in table 1. For example, such fermentation genes include those involved in auxine biosynthesis (such as Indoleactamide hydrolase, Tryptophan 2-monooxygenase), homolactic fermentation (such as L-lactate dehydrogenase), ethanol fermentation (such as alcohol dehydrogenase and pyruvate decarboxylase), acetate fermentation (such as pyruvate dehydrogenase, pyruvate formate-lyase, formate acetyltransferase), succinate fermentation (such as fumarate reductase), butanediol fermentation (such as acetolactate decarboxylase, butanediol dehydrogenase), fermentation and fermentation enzyme homologs, nitrate/nitrite uptake (such as nitrate ABC transporter, nitrate/nitrite transporter), ammonium uptake (such as ammonium transporter), phosphorous uptake (such as low-affinity inorganic P04 transporter, P04 ABC transporter, Na-dependent P04 transporter, phosphonate ABD transporter), cell division (such as FtsA, FStZ), glycolysis (such as fructose-bisphosphate aldolase, triosephosphate isomerase), DNA replication (such as replicative DNA helicase, DNA polymerase III), and fatty acid biosynthesis (such as 3-oxoacyl-ACP synthase, Acyl carrier protein ACP).

For example, the enriched fermentation capacity can comprise 0-5% over those normally present, 5-10% over those normally present, 10-15% over those normally present, 15-20% over those normally present, 20-25% over those normally present, 25-30% over those normally present, 30-35% over those normally present, 35-40% over those normally present, 40-45% over those normally present, 45-50% over those normally present, or greater than 50% over those normally present.

Aspects of the invention also comprise a method of improving soil quality. The term “improving soil quality” can refer to increasing the amount and/or availability of nutrients required by, or beneficial to plants, for growth. By way of example only, such nutrients include nitrogen, phosphorous, potassium, copper, zinc, boron and molybdenum. Also encompassed by the term “improving soil quality” is reducing or minimizing the amount of an element that can be detrimental to plant growth or development such as, for example iron and manganese. Thus, improving soil quality by use of enriched RAM thereby assists and promotes the growth of plants in the soil.

In embodiments, the method comprises cultivating in soil a plant that produces an enriched root associated microbiome (RAM), wherein the enriched RAM improves the soil quality. As described herein the plant can be a genetically engineered plant or, alternatively, the plant is not a genetically engineered plant, but is instead a natural plant or natural plant variant.

In other embodiments, the method comprises applying to soil an agricultural composition comprising an enriched root associated microbiome (RAM) or a portion thereof.

In embodiments, improved soil quality can be indicated by increased root yield, increased shoot yield, or increased crop yield.

In embodiments, the method comprises applying to the soil of a plant an agricultural composition comprising an enriched root associated microbiome (RAM) or a portion thereof. For example, the term “applying”, or “soil application” can refer to the application of a composition to the soil around a plant, where the intent is to affect the soil directly or to place the roots of the plant in contact with the substance. Substances applied through a soil application will or will not contact the foliage or will minimally contact the foliage. For example, the agricultural composition comprising the enriched RAM or portion thereof can be applied to the soil of a plant as an amendment in suspension to irrigation water, as dried pellets or as mixtures of microbes and soil particles.

In embodiments, the agricultural composition comprising an enriched root associated microbiome (RAM) is provided in an amount effective to increase plant growth, increase plant root yield, increase leaf foliage, increase plant shoot yield, increase crop yield, and/or improve soil quality.

As described herein, the enriched RAM comprises one or more viable microorganisms, one or more proteins, one or more sugars, one or more volatile organic compounds (VOCs), or any combination thereof.

For example, the enriched RAM can comprise a range of about 15% to 70% of Proteobacteria, about 3% to about 40% of Actinobacteria, about 0% to about 20% of Verrucomicrobia, about 0% to about 15% of Chloroflexi, about 0% to about 15% of Plastids, about 0% to about 15% of Nitrospirae, about 5% to about 40% of Bacteriodetes, about 5% to about 20% of Acidobacteria, about 0% to about 10% of Planctomycetes, about 0% to about 5% of Gemmatimonadetes, or about 0% to about 5% of Firmicutes.

In embodiments, the enriched RAM can comprise metabolites, such as metabolites of nitrogen metabolism. The term “metabolites” can refer to molecules or compounds formed from a metabolic process (e.g., metabolism, fermentation), including the molecules or compounds associated with compound degradation or elimination. For example, butanediol fermentation can refer to a type of fermentation in which organic compounds, such as sugars, are fermented (i.e., metabolized) with the production of metabolites such as 2,3-butanediol.

In embodiments, the enriched root associated microbiome (RAM) can be obtained by cultivating a genetically engineered plant in a soil composition for a period of time, and optionally removing the genetically engineered plant from the soil, thereby providing a composition comprising an enriched RAM.

In embodiments, the period of time that the length of time that the genetically engineered plant must be cultivated in soil to produce an enriched RAM can comprise one generation of cultivation. The skilled artisan will recognize that the length of time that the genetically engineered plant must be cultivated in soil to produce an enriched RAM can depend on the plant life cycle length.

Aspects of this invention are further drawn to a method of increasing plant growth or plant yield.

In embodiments, the method comprises cultivating in soil a plant that produces an enriched root associated microbiome (RAM), wherein the enriched RAM can increase plant growth or plant yield.

In other embodiments, the method comprises applying to soil an agricultural composition comprising an enriched root associated microbiome (RAM) or a portion thereof.

Aspects of the invention are also drawn towards an agricultural composition. The term “agricultural composition” can refer to a composition that can improve the rate of growth or health of plants, increasing the yields of plants or their fruits (and their plant parts, such as the roots or shoots), and/or improving or change the environments where the plants grow. In an embodiment, the agriculture composition supplements the soil with various nutrients for plant growth and produces the nutritional response from the plants. In another embodiment, the agricultural composition comprises one or more microbial species that can improve or change the environments where the plants grow. Other components of the agricultural composition can include, for example, a wetting agent, a binding agent, a filler, a preservative, a mineral, an adjuvant, a thickening agent, a bioprotector, an osmotic protectant, or an organic additive. In embodiments, the agricultural composition can be provided as a “dried” composition (such as a lyophilized composition).

In embodiments, the agricultural composition comprises an enriched root associated microbiome (RAM) or a portion thereof. As described herein, the enriched RAM can comprise one or more viable microorganisms, one or more proteins, one or more sugars, one or more volatile organic compounds (VOCs), or any combination thereof.

In embodiments, the enriched RAM can be obtained from a genetically engineered plant, such as that described herein. For example, the genetically engineered plant can be modified to overexpress a type 1 H⁺-pyrophosphatase. In embodiments, the genetically engineered plant is of the genus Arabidopsis. For example, the Arabidopsis plant is Arabidopsis thaliana. In addition to Arabidopsis thaliana, the genetically engineered plant as described herein can be any species of the genus Arabidopsis. For example, the Arabidopsis plant can be Arabidopsis Arenicola, Arabidopsis arenosa, Arabidopsis cebennensis, Arabidopsis croatica, Arabidopsis helleri, Arabidopsis lyrate, Arabidopsis neglecta, Arabidopsis pedemontana, Arabidopsis suecica, or Arabidopsis thaliana. In embodiments, the genetically engineered plant is Arabidopsis thaliana.

In embodiments, the agricultural composition can be produced by cultivating a genetically engineered plant in a soil composition such that the genetically engineered plant produces an enriched RAM within the soil composition, thereby providing the agricultural composition comprising an enriched RAM. The genetically engineered plant can be removed from the agricultural composition once the enriched RAM is produced.

In embodiments, the agricultural composition can comprise one or more metabolites, such as metabolites of nitrogen metabolism. For example, the agricultural composition can comprise 2, 3 butanediol.

In embodiments, the agricultural composition can comprise at least one plant nutrient. The term “nutrient or “plant nutrient” can refer to a mineral element considered essential for plant growth. For example, there minerals known to play essential roles in plant nutrition, include macronutrients, such as nitrogen (N), phosphorus (P), potassium (K), calcium (Ca), magnesium (Mg), sulfur(S), and micronutrients, such as boron (B), copper (Cu), iron (Fe), chloride (CI), manganese (Mn), molybdenum (Mo), and zinc (Zn). As such fertilizer is a plant nutrient. Secondary and micronutrients are also plant nutrients, which can include calcium, magnesium, sulfur, iron, manganese, copper, and zinc, including oxides thereof, salts thereof, ligands thereof, and combinations thereof.

As mentioned herein, the larger root systems can provide not only root exudates such as reduced carbon molecules, nitrogen rich compounds, hormones, but, without wishing to be bound by theory, can acidify soils, which can influence nutrient availability and use efficiency. Thus, higher P, N, K and other minerals, such as micronutrients, will be found. For example, roots can secrete protons that can free other cations (such as K, Mg, Ca, Fe) that are attached to negatively charged clay particles and bring them into the soil solution. Protons are also used to import negatively charged molecules (anions) such as nitrate, phosphate using proton anion cotransporters that localize at the plasma membranes of roots specially in root hairs.

Aspects of the invention are also drawn towards a method of manufacturing an agricultural composition comprising an enriched root associated microbiome (RAM). In embodiments, the method comprises cultivating a genetically engineered plant in a soil composition such that the genetically engineered plant produces an enriched RAM within the soil composition, thereby providing an agricultural composition comprising an enriched RAM.

In embodiments, the agricultural composition can be a soil-specific agricultural composition. For example, the soil-specific agricultural composition comprises an enriched RAM that is specific for the soil type. Referring to FIG. 5, for example, the enriched RAM is specific for the soil composition in which the genetically engineered plant is cultivated in.

In embodiments, the method comprises obtaining a soil sample and cultivating a genetically engineered plant in the soil sample such that the genetically engineered plant produces an enriched RAM within the soil sample, thereby providing a soil-specific agricultural composition comprising an enriched RAM. The term “soil sample” or “soil composition” can refer to a composition in which plants can grow. Soils can include a mixture of organic matter, minerals, gases, liquids and organisms that together support life. Optionally, the genetically engineered plant can be removed from the agricultural composition once the enriched RAM is produced.

In embodiments, the genetically engineered plant can be modified to overexpress a type 1 H⁺-pyrophosphatase. In embodiments, the genetically engineered plant is of the genus Arabidopsis. For example, the Arabidopsis plant is Arabidopsis thaliana.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Embodiments described herein comprising compositions and methods of using a specific type of transgenic (genetically modified) plant to select and produce a distinct assemblage of root-associated bacteria (root microbiome) from those naturally existing in the soil that can, upon transplant, improve the growth of other plants.

Embodiments described herein can be used in fields where plant productivity is important, such as crop agriculture to horticulture and environmental restoration.

Research has shown that:
1. proton pyrophosphate overexpressing mutants of the model plant Arabidopsis and crop plants show increased productivity with respect to their wild-type cognates.
2. These mutants develop a larger, distinct microbiome with differential microbial community composition from those of wild type plants
3. That these altered microbiomes are partly responsible for the more vigorous growth of the mutants
4. That the increased vigor of the mutants can be transferred to neighboring wild type plants

These findings lead us to validate whether an intervention in which wild type plants inoculated with microbiomes of the mutant plants can lead to improved plant growth of the non-transgenic plants, which we can demonstrate experimentally with respect to both root and shoot growth, thus providing us with a simple methodology to improve plant yield without the direct use of GMOs in the field.

Therefore, embodiments herein are drawn to a simple, natural methodology to provide growth-stimulating affordable additives in activities requiring plant cultivation, such as crop agriculture, horticulture and environmental restoration.

Embodiments herein are easy of produce, use and deliver. Further, embodiments herein can use additives (such as microbiomes) obtained from microbes existing in the soil. Embodiments herein comprise minimal technological requirements for production and implementation. Further, environmental impact of the applications are negligible in comparison to alternatives such as fertilization.

Embodiments herein are based on complex mixtures of microorganisms, rather than the use of cultivated plant-growth promoting bacteria reared in the laboratory. All additives can be pre-selected to thrive in the plant root environment.

### Example 2

Embodiments described herein comprise a new methodology to enhance the productivity of wild type crops by transferring or transporting useful/robust phenotypes from a neighboring high yielding mutant species via root associated microbiome (RAM) of the mutant species. This distinct/unique variety of microbiome which confers these robust phenotypes to mutant species are in the soil and thus requires minimal technology to improve the productivity of the wild type plants.

Type 1 proton pyrophosphatase overexpressing transgenic Arabidopsis plants demonstrate enhanced root and shoot biomass production along with improved salinity and drought endurance.

These high yielding phenotypes can be easily transferred from the mutant species to neighboring wild type species and this is mediated by specific and functional root associated microbiome (RAM) of the mutant species not found in wild type variety.

This transfer of specific root associated microbiome from mutant to wild type is through production of bacterial fermentation product of 2,3 butanediol by root exudate of mutant variety.

This represents a very simple method of adding natural additives involving least technological requirement and minimum adverse environmental impact that is associated with use of excessive fertilizers.

### Example 3

Microbiome-Mediated Phenotype Transfer from Transgenic Plants

The expression of an organism's genes determines its own characteristics in a given environment. And yet, we found that the phenotype of transgenic plants genetically modified to enhance yield (1) transfers readily to neighboring wild type plants, challenging that tenet. We show that the transgenic's phenotype extends to its root-associated microbiome (RAM), which is larger, of different specific composition and functional potential than the wild type's, as a result of altered root exudation. We provide evidence that the RAM not only helps determine the transgenic's robust phenotype but also mediates its transfer to neighboring plants through the production of fermentative 2,3-butanediol.

Thus, the root-associate microbiome of transgenic plants not only helps determine their phenotype, but can elicite its export to neighboring wild type plants

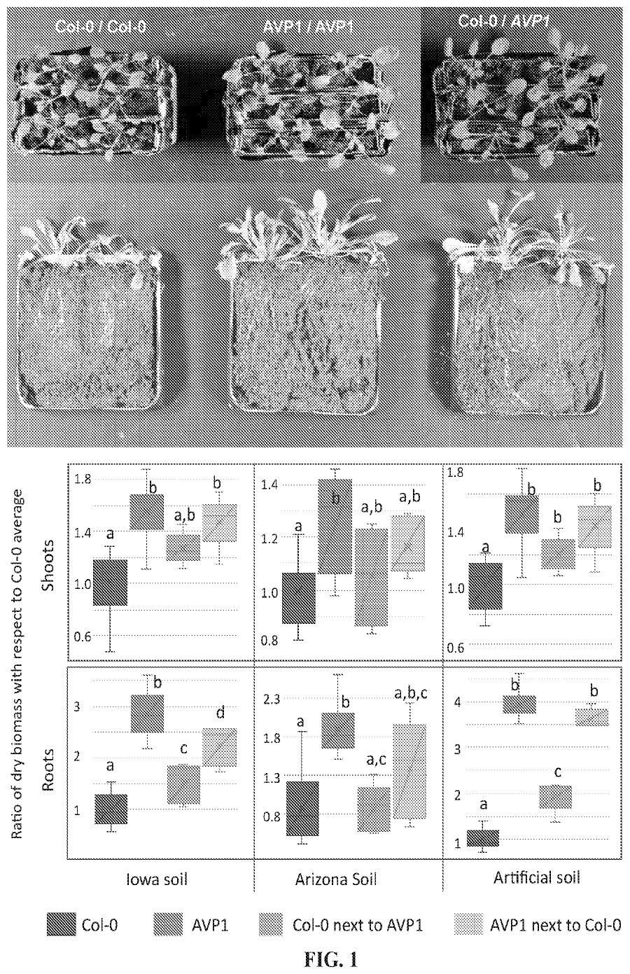

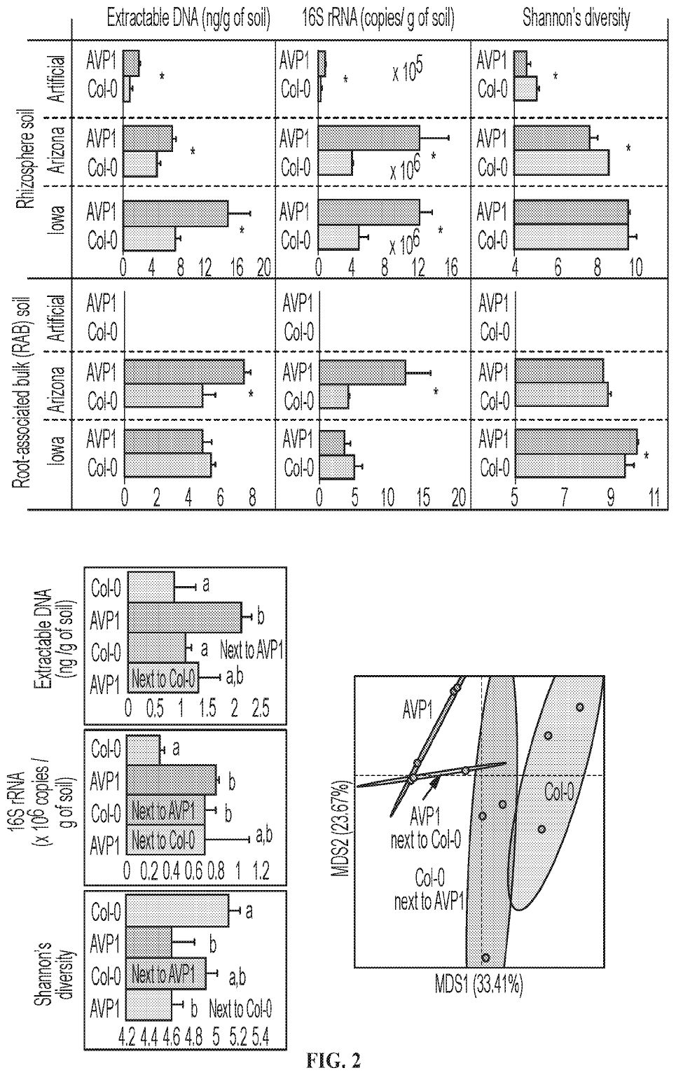

Genetic modification of crop genotypes to provide for phenotypes is standard in agriculture (2), based on the correspondence between genotype and observed phenotype (3). The gene for type I H⁺-pyrophosphatase from Arabidopsis thaliana has been identified as a yield enhancement determinant whose overexpression triggers production of more, larger leaves and root biomass over the wild type (4, 5), which, without wishing to be bound by theory, is due to enhanced carbon translocation to the root tissues. This in turn promotes nutrient and water acquisition capacity. These mutants also possess increased salinity and drought tolerance (6, 7). Similar differential phenotypes of H⁺-PPase overexpressing transgenics are seen in crop plants including rice, barley, tobacco, cotton, alfalfa, maize, and wheat (8). Another very conserved phenotype associated with these genetically modified plants is an increased acidification of the soil surrounding roots (7, 9, 10). Because all of these traits are governed by plant physiological mechanisms, we were surprised to find that the phenotype of A. thaliana 35S::AVP1-1 (with ubiquitous expression of H⁺-PPase; hereafter, AVP1) can be partly transferred to nearby wild-type plants (A. thaliana Col-0; hereafter Col-0) as seen in FIG. 1.

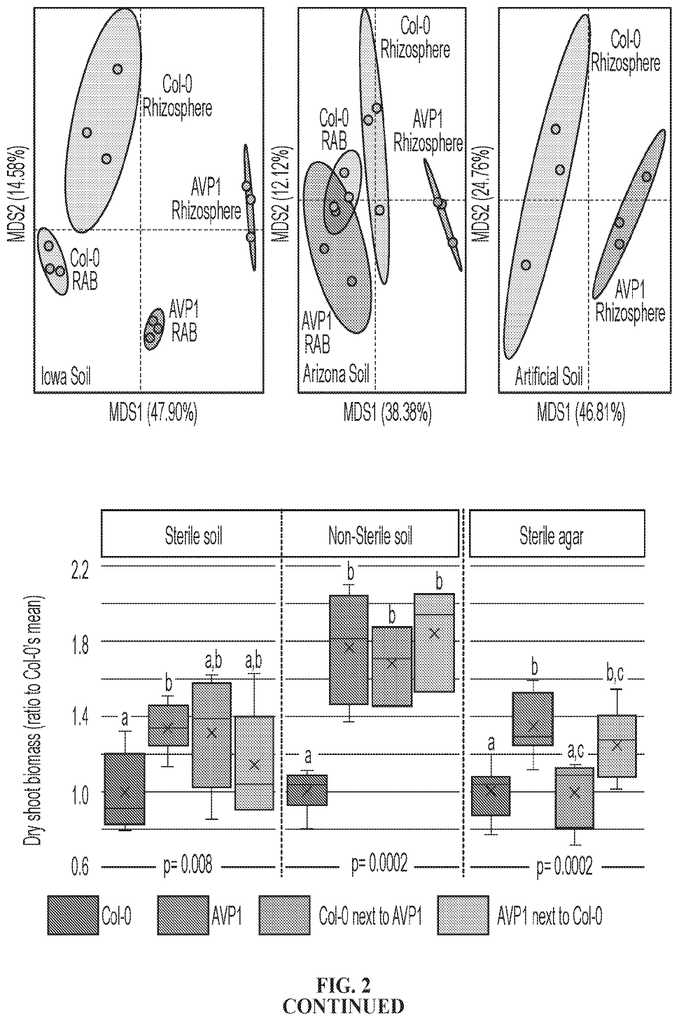

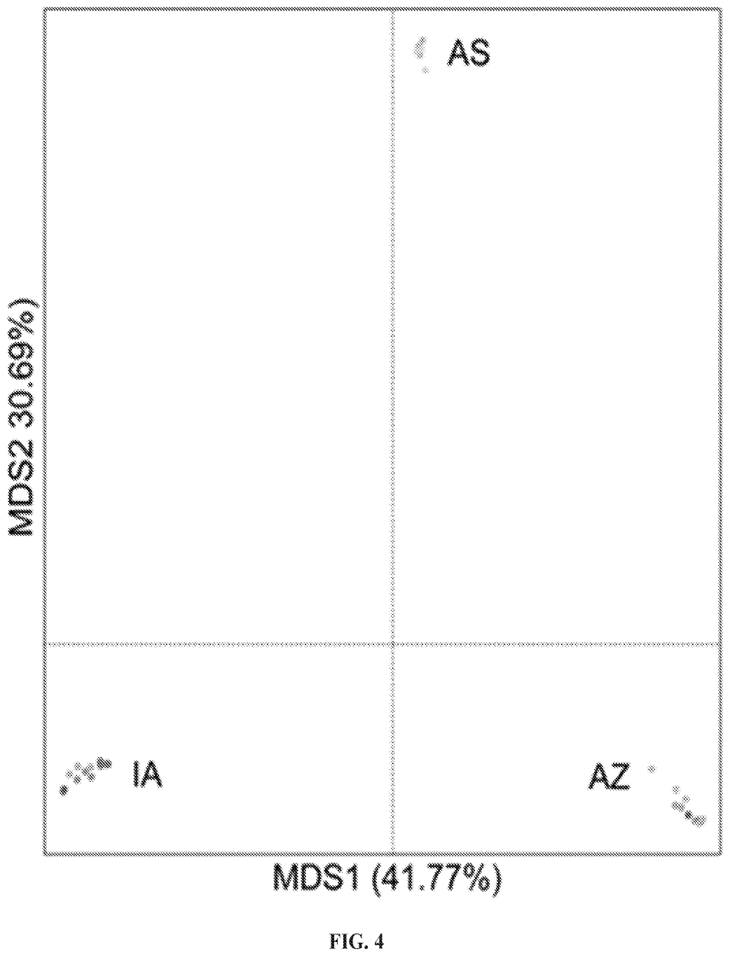

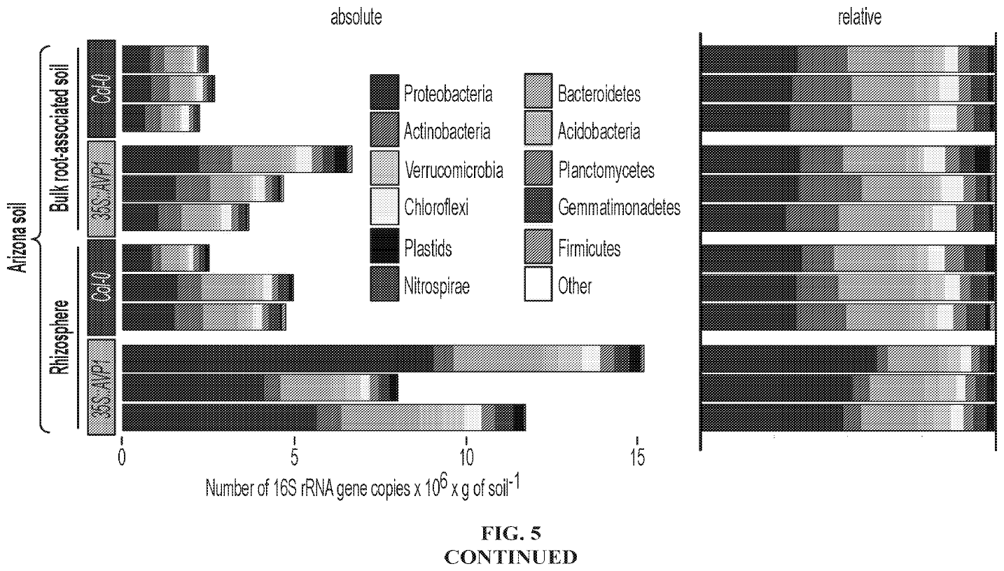

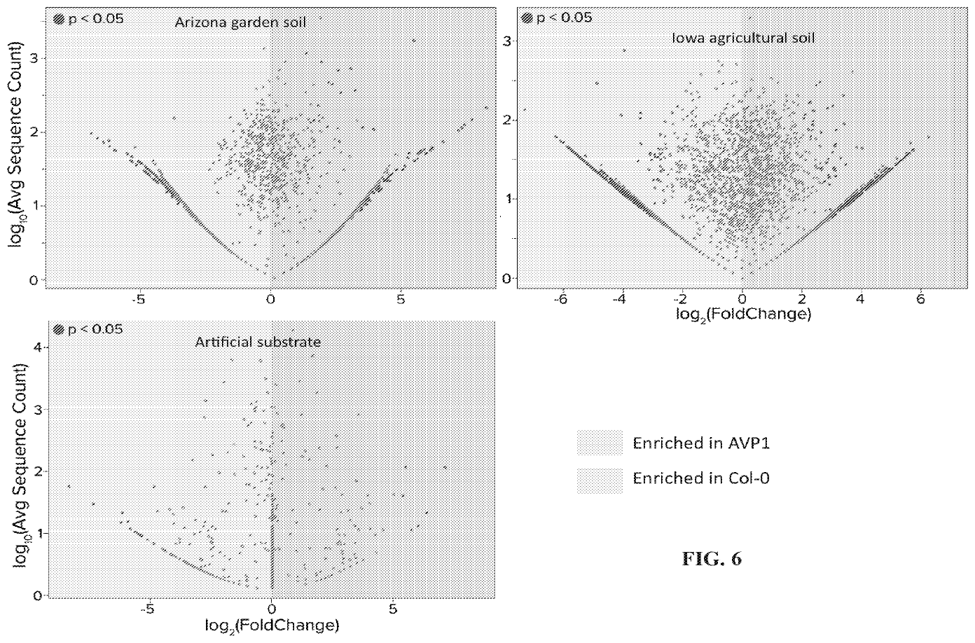

The transfer occurred in artificial ceramic substrates (Artificial Soils) and in two natural soils of differing potential fertility: an aridisol-based garden soil from Arizona (Arizona Soil), and a black agricultural soil from Iowa (Iowa Soil). When homogenetic pairs were grown in of these soils, AVP1 genotypes always yielded significantly more biomass of shoots and roots than homogenetically grown Col-0 pairs (post-ANOVA t-test corrected for multiple comparisons p<0.001). But when grown as heterogenetic pairs, shoot and root mean yields of Col-0 and AVP1 plants were always intermediate to those of homogenetically grown pairs, although not always significantly different. At least one of the two genotypes in heterogenetic pairs differed in yield from those of its counterpart grown homogenetically, or non-different from those of their opposite genotype. It was as if the genotype of one plant was partly determining the phenotype of its neighbor. This observation implicates factors external to the plant as determinant of phenotype. Several indirect lines of evidence point to the root-associated microbiome (RAM) as a suspect. First, evidence indicates that plant-root microbiome interactions determine outcomes, (11, 12), and evidence indicates that microbiome compositional blending in mixed cropping of plant varieties (13). Second, the enhanced photosynthate transport to roots, and the acidification of the soil surrounding the root system, are factors known to affect soil microbiomes: pH is the most important environmental factor driving soil microbial diversity (l4) and long standing principles (15) indicate that increased root exudation will result in higher microbial biomass. Modifications in exudate composition in fact do shift RAM composition (16, 17). Even genetically close accessions of A. thaliana (18) and crop plants (13) sustain distinct RAMs. Thus, without wishing to be bound by theory, part of the phenotypic robustness shown by our transgenics can be traced back to a more densely populated or functionally modified RAM. To establish if the mutant plant genotype affected its RAM, effectively extending the differential phenotype beyond the plant itself, we compared basic traits of Col-0 and AVP1 RAMS challenged by growth in soils that support divergent microbiomes (FIG. 4), distinguishing microbiomes closely associated with the roots (Rhizosphere RAMs) and those loosely associated with the root systems (Bulk Root-Associated microbiomes, or BRAMs) (19). The rhizosphere RAMs of AVP1 were indeed significantly more populated than those of Col-0, regardless of soil substrate, both in terms of extractable DNA and concentration of 16S rRNA gene copies determined by qPCR (FIG. 2). A fourfold increase in root biomass (FIG. 1) compounded with a threefold difference in microbiome concentration implies that the rhizosphere RAM of AVP1 transgenics was close to an order of magnitude larger than that of the wild type's. Rhizosphere RAMS were also significantly less diverse in Arizona and Artificial soils, based on amplicon sequence analyses of 16S rRNA genes, although not in naturally rich Iowa soils. Further, on the basis of compositional similarity analyses, rhizosphere RAM bacterial communities (FIG. 2) of Col-0 and AVPI differed significantly from each other, regardless of soil substrate. Thus, the differential phenotype associated with the H⁺-PPase overexpressing mutation robustly extended to the rhizosphere microbiome across a variety of soils that contain highly divergent RAMs. However, changes in community composition between plant genotypes did not involve the same bacterial types in the different soils: while in Arizona soils AVP1 recruited more Proteobacteria and less Actinobaceria than Col-0, in Iowa soils it recruited more Bacteriodetes and Verrucomicrobia to the detriment of Actinobacteria. Yet in artificial soils, which were initially devoid of a soil microbiome, AVPI recruited Actinobacteria and Bacteriodetes to the detriment of Proteobacteria (FIG. 5 and FIG. 6). Apparently, the differential selection of microbial types is exerted on the available biodiversity (20, 21), which differed significantly among soils. The effects seen on rhizosphere RAMs in some cases extended to the BRAMs (FIG. 2), in composition or in abundance, or both, except in the artificial soil where the BRAM was underdeveloped. In these artificial soils, however, one can trace and separate the roots according to plant genotype when Col-0 and AVP1 were grown as heterogenetic pairs, enabling an assessment of the transferability of RAM characteristics: plants grown with the opposite genotype developed RAMS that were intermediate in composition and often in diversity and population density to those grown with their own genotype (FIG. 2). The rhizosphere RAM phenotypes, like the plant phenotypes, were thus also partly transferable.

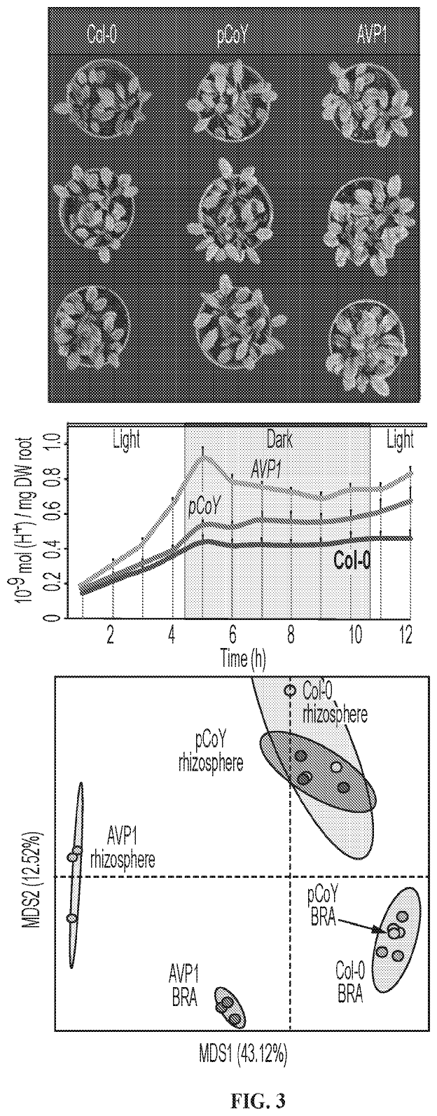

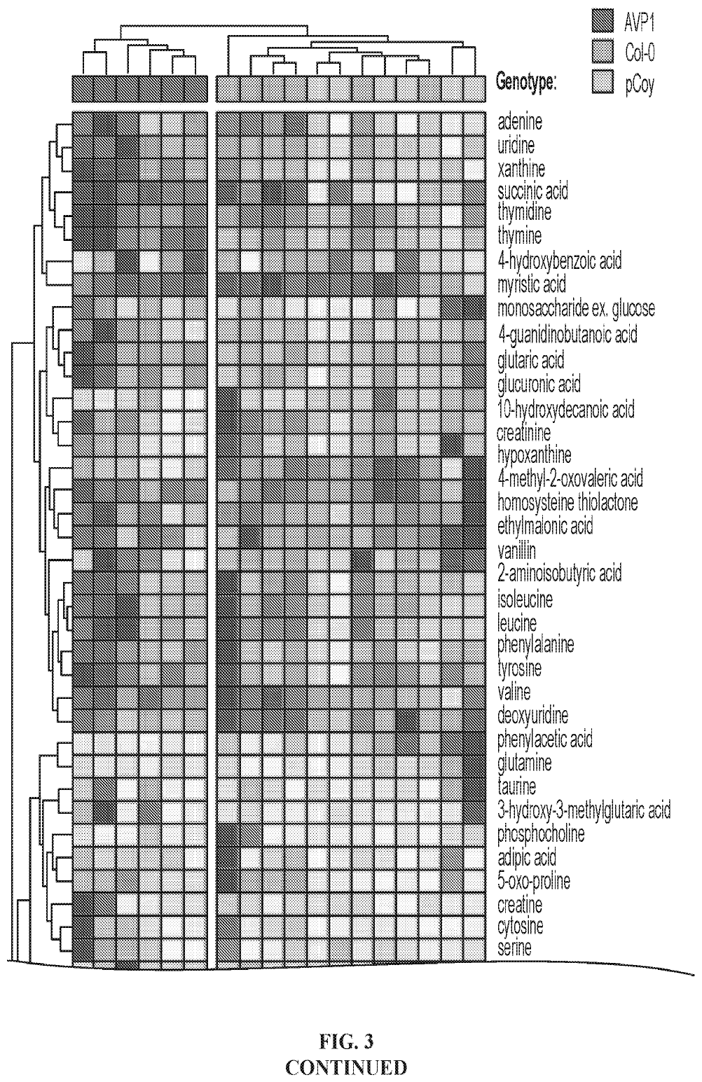

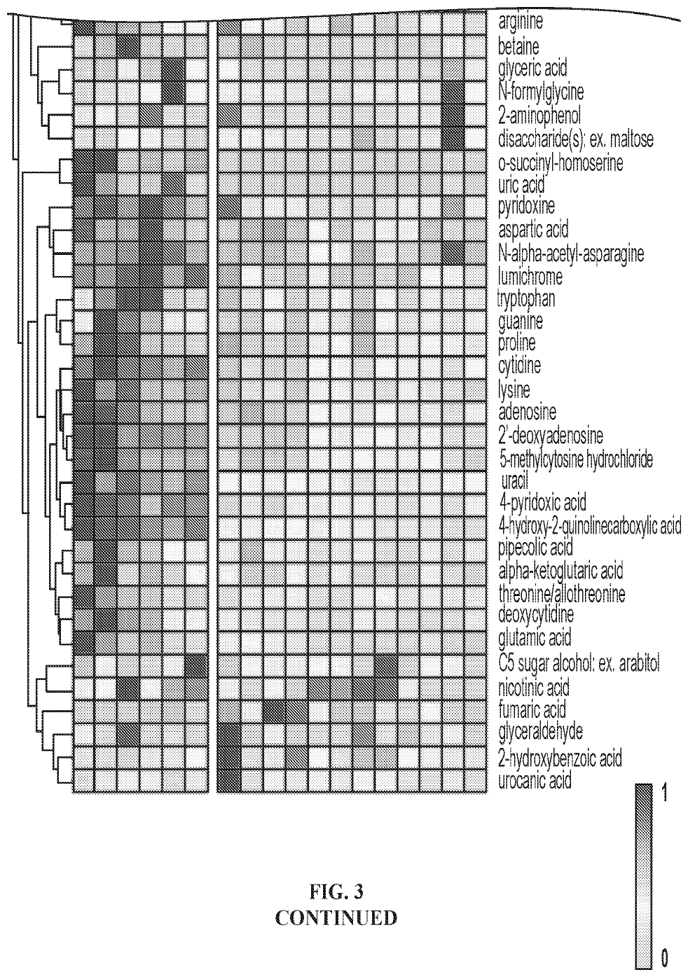

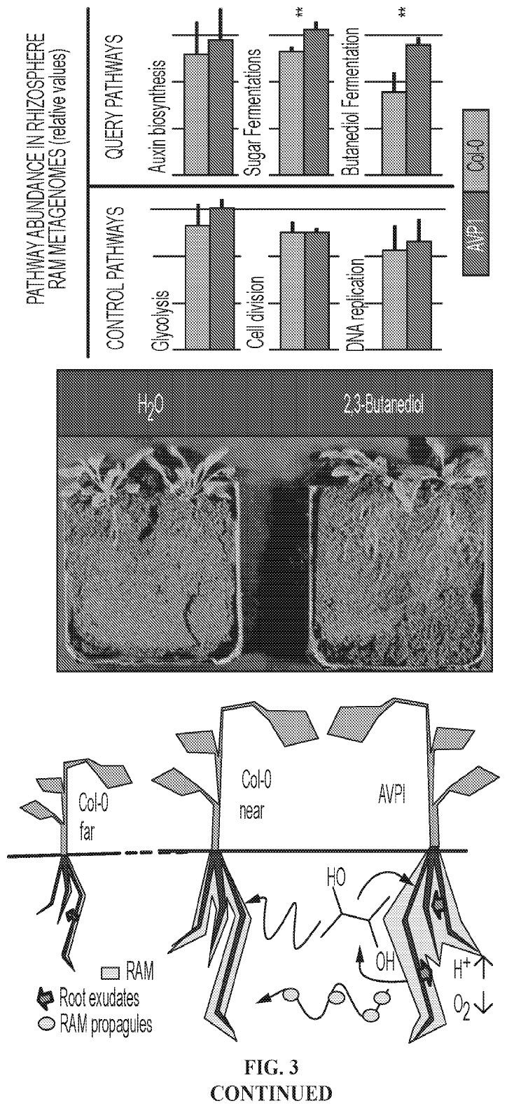

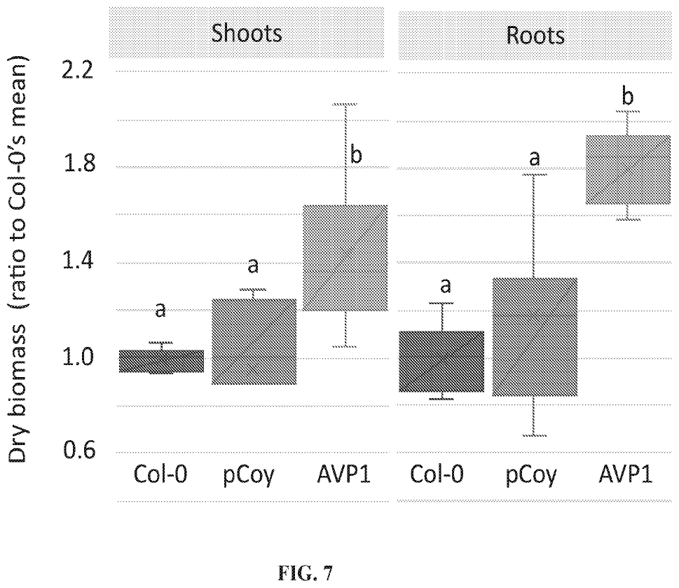

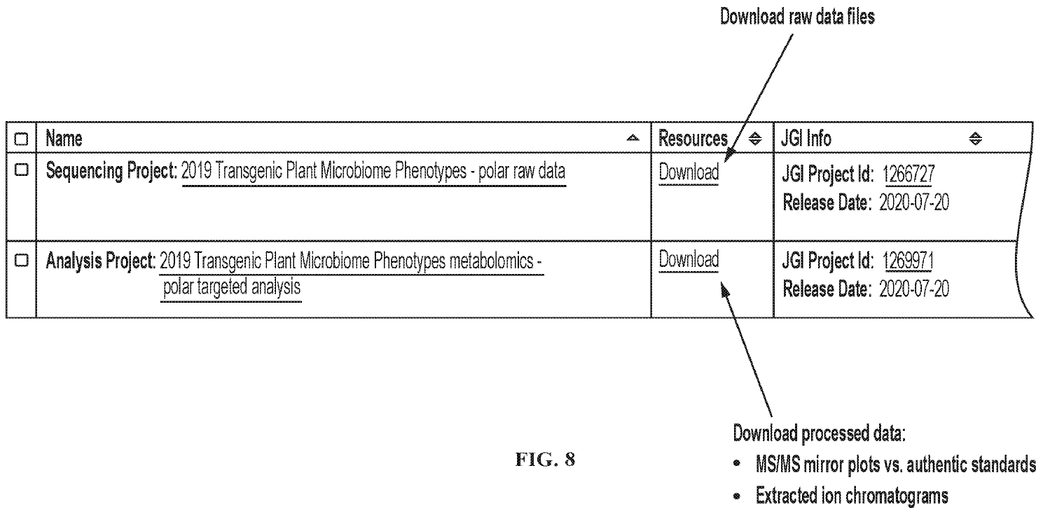

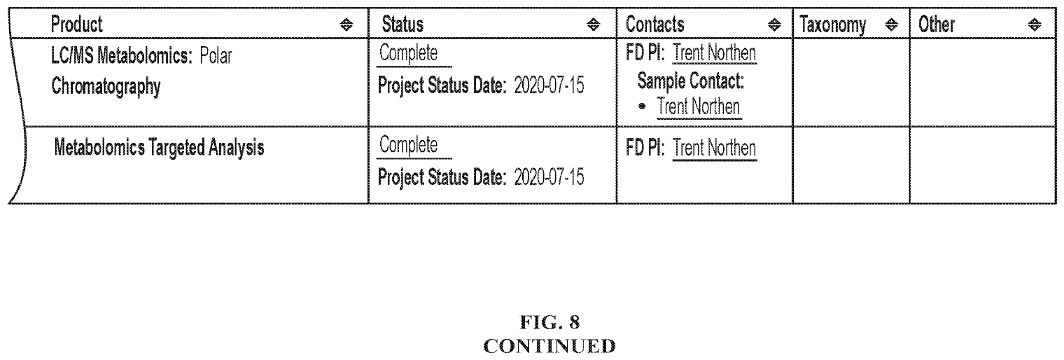

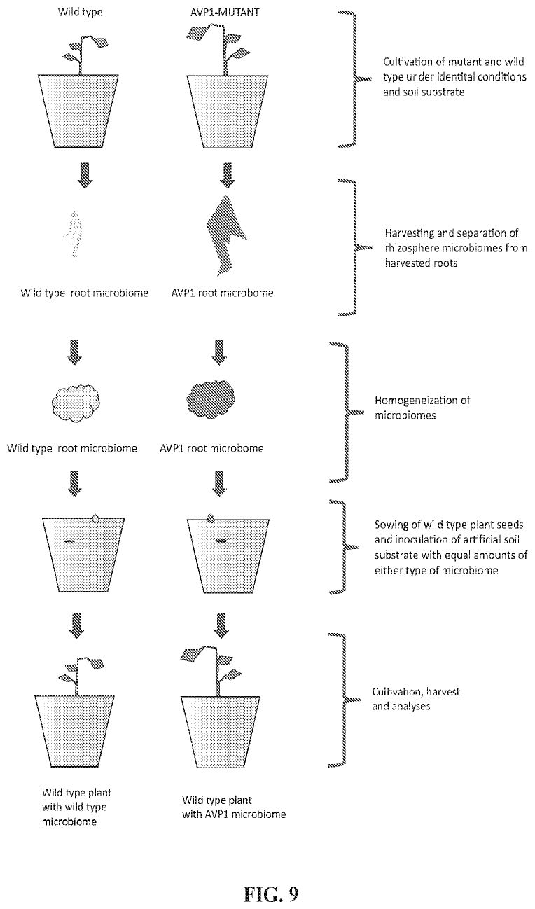

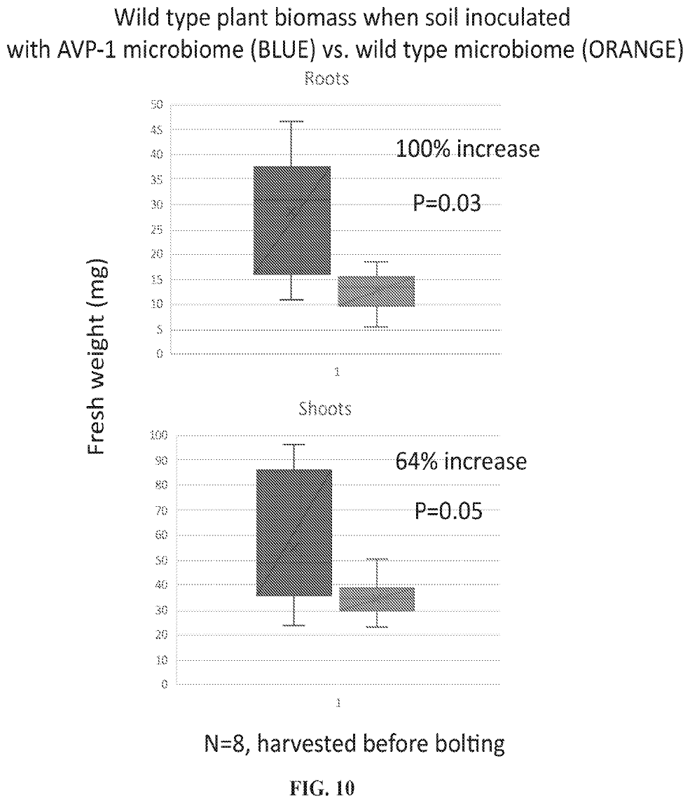

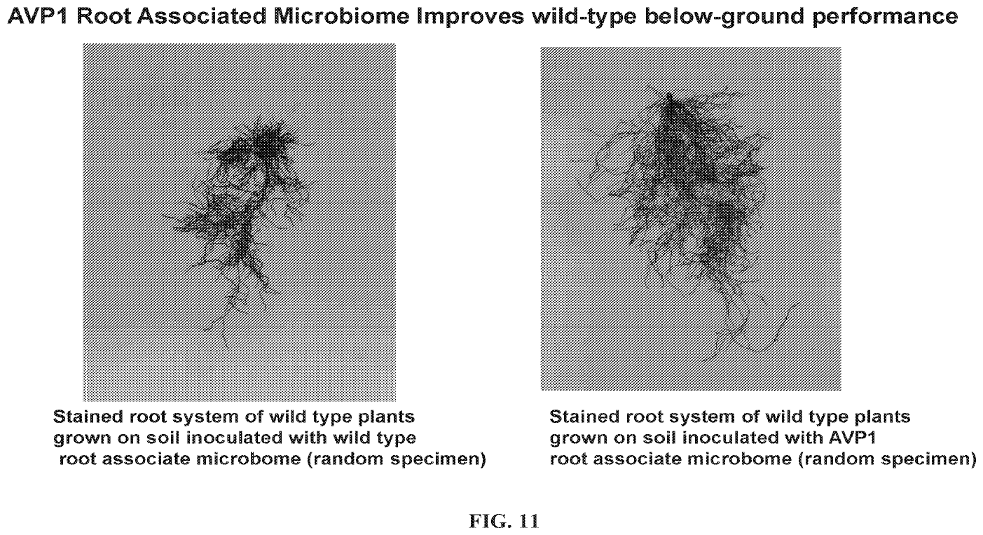

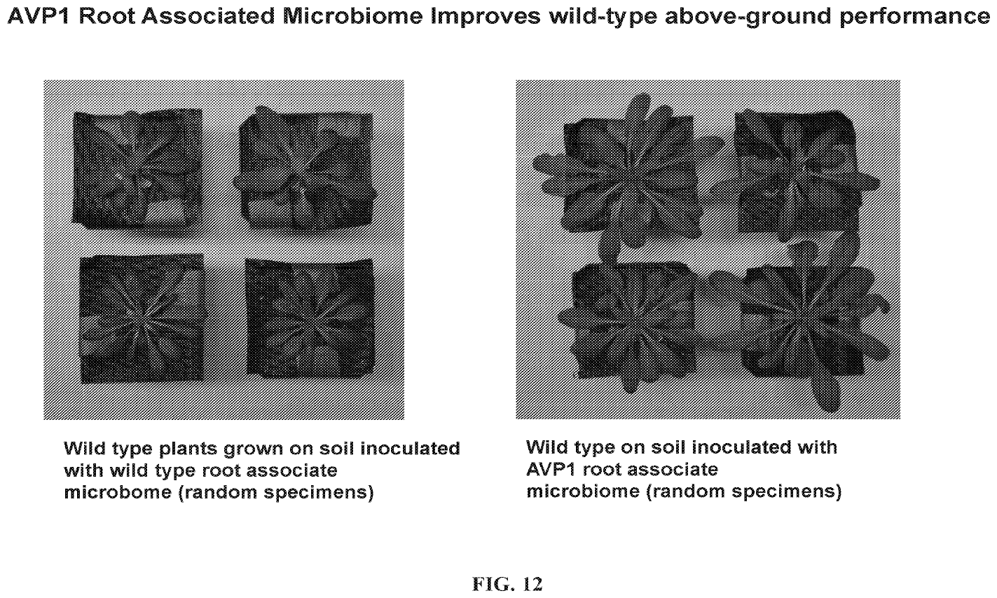

To determine how the plant genotype can be made extensive to the RAM, we made use of a second mutant plant (pCoYMV::AVP1; pCoY hereafter), which overexpresses the H+-PPase in companion cells and sieve elements of the plant's phloem (22). The yields of pCoY plants were not significantly different from Col-0 's (FIG. 3 and FIG. 7), but it still acidified the soil more than the wild type, albeit not as strongly as AVPI's (FIG. 3). We found that the composition (FIG. 3) of pCoY RAMs did not differ from that of Col-0's, in rhizosphere or BRAM fractions, but was significantly different from those of AVP1. If acidification were the major factor driving RAM community changes in transgenics, one would have expected shifts in pCoY's RAM similar to those detected for AVP1, but we did not detect any (FIG. 3). We then assessed a second potential driver: root exudates. We determined the root exudate profiles, or exometabolome (23), of the three genotypes when grown under sterility on agar-solidified medium. The exometabolomes of replicate genotypes (FIG. 3; see FIG. 4 for identification details) were most self-similar, those of pCoY being indistinguishable from those of Col-0. A set of 18 compounds were excreted by AVP1 (ANOVA+Tukey's test, p<0.05), all nitrogenous metabolites, with nitrogenous bases, amino acids and their derivatives being prominent, consistent with the shift toward increase nitrogen metabolism of AVP1 (24). Without wishing to be bound by theory, this differential excretion can exert a functional enrichment on available soil microbial biodiversity. To validate this, we conducted a comparative analysis of the rhizophere RAM metagenomes of COL-0 and AVPI plants. Indeed, consistent with AVP1's nitrogenous root excretions, its RAM metagenome was comparatively enriched in uptake genes for reduced nitrogen and depleted in those for nitrate uptake, while the proportion of genes for universal functions such as cell division were not different (Table 1). We then validated whether microbiome-produced plant hormones is the link between microbiome and plant phenotype. Because of the abundance of tryptophan in the exudates of AVP1, a candidate was auxin (indole acetic acid), but the genes for its synthesis were not significantly enriched in AVP1 RAMs. Alternatively, the volatile 2,3 butanediol, a product of bacterial fermentation, can also act as a growth inducing hormone (25). Indeed, key genes for major fermentation pathways were enriched in AVP1 RAMs, consistent with a denser RAM that can easily become oxygen-limited. Among them, 2,3 butanediol fermentation was the most enriched, consistent with it being a pathway under acidic conditions (26). Metagenomics thus implicated 2,3 butanediol production as the missing link. To test this strictly, we grew wild type plants in the presence of 2,3 butanediol. This indeed resulted in AVP1-like, high yield phenotypes not only in shoots as previously shown (25), but also in root development (FIG. 3), providing a ready mechanistic explanation (see diagram in FIG. 3) for both single-plant phenotype enhancement and cross-plant phenotype transferability, given its volatility.

That the genotype of one plant modifies another plant's expression of its own genotype, as we describe here, has implications for the use of transgenic plants, since part of their vigor can be expended in promoting growth of neighboring plants, perhaps including unwanted species. That such proximal phenotype transfer can occur in a single generation is remarkable. It materialized faster than other potential secondary effects based on plant evolutionary processes (27) or transgene introgression (28). Also, without wishing to be bound by theory, it has cumulative effects in the soil microbiome after repeated cycles of growth, as the microbiomes become progressively more enriched in 2, 3-butanediol fermenters, perhaps also extending its spatial range. Thus, we will include assessments of RAM effects in the risk and benefit evaluation of transgenic plants.

### REFERENCES CITED IN THIS EXAMPLE

1. R. A. Gaxiola, C. A. Sanchez, J. Paez-Valencia, B. G. Ayre, J. J. Elser, Genetic manipulation of a “vacuolar” H (+)-PPase: from salt tolerance to yield enhancement under phosphorus-deficient soils. Plant Physiol 159, 3-11 (2012).
2. W. Klümper, M. Qaim, A meta-analysis of the impacts of genetically modified crops. PLOS One 9, e111629-e111629 (2014).
3. W. Johannsen, The Genotype Conception of Heredity. The American Naturalist 45, 129-159 (1911).
4. J. Li et al., Arabidopsis H⁺-PPase AVP1 regulates auxin-mediated organ development. Science 310, 121-125 (2005).
5. N. Gonzalez et al., Increased leaf size: different means to an end. Plant Physiol 153, 1261-1279 (2010).
6. R. K. Schilling et al., Expression of the A rabidopsis vacuolar H⁺-pyrophosphatase gene (AVP 1) improves the shoot biomass of transgenic barley and increases grain yield in a saline field. Plant biotechnology journal 12, 378-386 (2014).
7. A.-K. Bao et al., Overexpression of the Arabidopsis H⁺-PPase enhanced resistance to salt and drought stress in transgenic alfalfa (Medicago sativa L.). Plant Science 176, 232-240 (2008).
8. R. A. Gaxiola, K. Regmi, K. D. Hirschi, Moving On Up: H⁺-PPase Mediated Crop Improvement. Trends in Biotechnology 34, 347-349 (2016).
9. H. Yang et al., Enhanced phosphorus nutrition in monocots and dicots over-expressing a phosphorus-responsive type I H⁺-pyrophosphatase. Plant biotechnology journal 5, 735-745 (2007).
10. G. A. Pizzio et al., Arabidopsis type I proton-pumping pyrophosphatase expresses strongly in Phloem, where it is required for pyrophosphate metabolism and photosynthate partitioning. Plant Physiol 167, 1541-1553 (2015).
11. G. Berg, K. Smalla, Plant species and soil type cooperatively shape the structure and function of microbial communities in the rhizosphere. FEMS Microbiology Ecology 68, 1-13 (2009).
12. K. Schlaeppi, D. Bulgarelli, The Plant Microbiome at Work. Molecular Plant-Microbe Interactions 28, 212-217 (2014).
13. A. Horner, S. S. Browett, R. E. Antwis, Mixed-Cropping Between Field Pea Varieties Alters Root Bacterial and Fungal Communities. Sci Rep-Uk 9, 16953 (2019).
14. N. Fierer, R. B. Jackson, The diversity and biogeography of soil bacterial communities. Proceedings of the National Academy of Sciences of the United States of America 103, 626-631 (2006).
15. L. Hiltner, Über neuere Erfahrungen und Probleme auf dem Gebiet der Bodenbackteriologie und unter besonderer Berucksichtigung der Grundung und Brache. Arbeiten der Deutschen Landwirtschaftlichen Gesellschaft 98, 59-78 (1904).
16. H. P. Bais, T. L. Weir, L. G. Perry, S. Gilroy, J. M. Vivanco, The role of root exudates in rhizosphere interactions with plants and other organisms. Annu. Rev. Plant Biol. 57, 233-266 (2006).
17. S. L. Lebeis et al., Salicylic acid modulates colonization of the root microbiome by specific bacterial taxa. Science 349, 860-864 (2015).
18. S. Monchgesang et al., Natural variation of root exudates in Arabidopsis thaliana-linking metabolomic and genomic data. Sci Rep-Uk 6, (2016).
19. C. D. Barillot, C.-O. Sarde, V. Bert, E. Tarnaud, N. Cochet, A standardized method for the sampling of rhizosphere and rhizoplan soil bacteria associated to a herbaceous root system. Annals of microbiology 63, 471-476 (2013).
20. F. Liu et al., Soil indigenous microbiome and plant genotypes cooperatively modify soy bean rhizosphere microbiome assembly. BMC Microbiology 19, 201 (2019).
21. K. Zhalnina et al., Dynamic root exudate chemistry and microbial substrate preferences drive patterns in rhizosphere microbial community assembly. Nature microbiology 3, 470 (2018).
22. A. S. Khadilkar et al., Constitutive and companion cell-specific overexpression of AVP1, encoding a proton-pumping pyrophosphatase, enhances biomass accumulation, phloem loading and long-distance transport. Plant physiology 170, 401-414 (2016).
23. R. Baran et al., Exometabolite niche partitioning among sympatric soil bacteria. 6, 8289 (2015).
24. N. Gonzalez et al., Increased leaf size: different means to an end. Plant physiology 153, 1261-1279 (2010).
25. C.-M. Ryu et al., Bacterial volatiles promote growth in Arabidopsis. Proceedings of the National Academy of Sciences 100, 4927-4932 (2003).
26. R. Van Houdt, A. Aertsen, C. W. Michiels, Quorum-sensing-dependent switch to butanediol fermentation prevents lethal medium acidification in Aeromonas hydrophila AH-IN. Research in microbiology 158, 379-385 (2007).
27. J. A. Lau, J. T. Lennon, Rapid responses of soil microorganisms improve plant fitness in novel environments. Proceedings of the National Academy of Sciences of the United States of America 109, 14058-14062 (2012).
28. C. N. Stewart, M. D. Halfhill, S. I. Warwick, Transgene introgression from genetically modified crops to their wild relatives. Nature Reviews Genetics 4, 806-817 (2003).
29. R. A. Gaxiola et al., Drought- and salt-tolerant plants result from overexpression of the AVP1 H⁺-pump. Proceedings of the National Academy of Sciences 98, 11444-11449 (2001).
30. G. A. Pizzio, K. Regmi, R. Gaxiola, Rhizosphere Acidification Assay. Plant physiology, (2015).
31. D. S. Lundberg et al., Defining the core Arabidopsis thaliana root microbiome. Nature 488, 86 (2012).
32. M. Porcar et al., Microbial Ecology on Solar Panels in Berkeley, CA, United States. Front Microbiol 9, 3043-3043 (2018).
33. B. P. Bowen, T. R. Northen, Dealing with the unknown: metabolomics and metabolite atlases. Journal of the American Society for Mass Spectrometry 21, 1471-1476 (2010).
34. T. Hothorn, F. Bretz, P. Westfall, Simultaneous inference in general parametric models. Biometrical Journal: Journal of Mathematical Methods in Biosciences 50, 346-363 (2008).
35. R. Kolde, Pheatmap: pretty heatmaps. R package version 1, (2012).
36. E. Couradeau, A. Giraldo-Silva, F. De Martini, F. Garcia-Pichel, Spatial segregation of the biological soil crust microbiome around its foundational cyanobacterium, Microcoleus vaginatus, and the formation of a nitrogen-fixing cyanosphere. Microbiome 7, 55 (2019).
37. D. Roush, E. Couradeau, B. Guida, S. Neuer, F. Garcia-Pichel, A new niche for anoxygenic phototrophs as endoliths. Appl. Environ. Microbiol. 84, e02055-02017 (2018).
38. E. Bolyen et al., Reproducible, interactive, scalable and extensible microbiome data science using QIIME 2. Nature biotechnology 37, 852-857 (2019).
39. B. J. Callahan et al., DADA2: high-resolution sample inference from Illumina amplicon data. Nature methods 13, 581 (2016).
40. J. Bethany, A. Giraldo-Silva, C. Nelson, N. N. Barger, F. Garcia-Pichel, Optimizing production of nursery-based biological soil crusts for restoration of arid land soils. Applied and environmental microbiology, AEM. 00735-00719 (2019).
41. D. Li, C.-M. Liu, R. Luo, K. Sadakane, T.-W. Lam, MEGAHIT: an ultra-fast single-node solution for large and complex metagenomics assembly via succinct de Bruijn graph.
Bioinformatics 31, 1674-1676 (2015).
42. D. Hyatt et al., Prodigal: prokaryotic gene recognition and translation initiation site identification. BMC bioinformatics 11, 119 (2010).
43. W. Li, A. Godzik, Cd-hit: a fast program for clustering and comparing large sets of protein or nucleotide sequences. Bioinformatics 22, 1658-1659 (2006).
44. B. Li, C. N. Dewey, RSEM: accurate transcript quantification from RNA-Seq data with or without a reference genome. BMC bioinformatics 12, 323 (2011).
45. D. H. Huson, S. Mitra, H.-J. Ruscheweyh, N. Weber, S. C. Schuster, Integrative analysis of environmental sequences using MEGAN4. Genome research 21, 1552-1560 (2011).
46. R. C. Team. (Vienna, Austria, 2013).

### Materials and Methods

Plant lines. Genetically modified plant lines were available from prior research. Briefly, “Col-0” was the wild type, “AVP1” refers to ubiquitous overexpression of the AVP1 proton pyrophosphatase through insertion of a 35S::AVP1 cassette (29), and pCoY refers to phloem-specific expression of the proton pyrophosphatase through insertion of a [pCoYMV]::AVP1 cassette endowed with the Commelina Yellow Mottle virus promoter (10).

Plant growth. All seeds were surface-sterilized by a treatment of 10 min in 2% sodium hypochlorite and 0.01% Tween 20, followed by three washes in sterile deionized water (2 min each). For sowing in natural soil pots, seeds were stratified in the dark at 4° C. for one day, pots were spatially randomized and placed in growth chambers providing short days of 8 h light (300-400 1×) at 21° C. and 16 h dark at 18° C. Soil moisture was maintained with bi-weekly irrigations using deionized water and a scale to adjust the weight to the field capacity of approximately 80%. During the initial stage seedlings were fertilized one time using half-strength Murashige & Skoog (MS; GIFCO BRL) without vitamins, pH 5.8. For growth on plates, agar or agarose (0.9% w/v) was prepared with half-strength MS medium and 1% (w/v) sucrose, seeds stratified as described herein, and then plates incubated at a 45° angle from the horizontal for one week, then transferred to sucrose-free plates for 2 weeks. For growth on artificial soils, seedlings from agar plates were transferred to open deep square plates or completely enclosed plastic boxes (non-sterile or sterile growth conditions, respectively) containing packed substrate, and incubated in the growth chamber as described herein. Soils used were: i) Arizona soil, an aridisol-derived garden soil from a local yard, ii) Iowa soil, a dark organic agricultural soil, and iii) Artificial soil, was a commercial inorganic-profile porous ceramic substrate (Turface®) that contained no detectable original soil microbiome.

Acidification assay. The acidification assay was performed according to Pizzio et al. (30). Seeds were germinated and grown aseptically on vertical agar plates for 7 days. Then, seedlings (ten per point) were transplanted to flasks with 4 ml of liquid media (half strength MS, 1% (w/v) Sucrose) and were grown for two weeks. Roots were washed with the assay solution (quarter strength MS and 2 mM MES buffer pH 6.8) for 5 minutes. Acidification assay was performed in fresh assay solution through a 12 h dark/12 H light cycle. Protons released relative to fresh root biomass were derived from pH measurements.

Plant harvesting and root-associated microbiome fractionation. For plant biomass determinations, shoots and roots from five-week old plants were harvested, separated, cleaned and dried at 60° C. for gravimetric determination of dry biomass data. For root-associated microbiome fractionation and isolation, roots were harvested, and the soil was divided into three distinct fractions as previously described (31): loose soil, bulk soil and rhizosphere soil. Loose soil was left behind on the pots on harvesting and discarded. Bulk, root associated soil was manually removed from the roots by kneading and shaking with sterile gloves (sprayed with 70% EtOH) and by patting roots with a sterile (flamed) metal spatula. The rhizosphere fraction was obtained by placing roots freed of bulk soil in a sterile 50 ml tube containing 25 ml phosphate buffer (per litre: 6.33 g of NaH₂PO₄·H₂O, 16.5 g of Na₂HPO₄. 7H₂O, and Tween 20 [0.01% v/v]), and vortexing at maximum speed for 15 s. This process was repeated until the roots were completely clear. The turbid solution was gravity filtered through a glass funnel with a filter paper into a new 50-ml tube to remove broken plant. parts and large sediments. The turbid solution was then centrifuged for 15 min at 5,000. After discarding the supernatant, the rhizosphere pellet was stored at −20° C. until further processing.

Root exudate collection and metabolomics. After four weeks growing in agarose MS nutrient media, collected root exudates were filtered using nylon filters of pore size 0.45 μm (Millipore, MA) to remove root sheathing and cells, and stored at −80° C. until further analyses. Sterile techniques were used throughout and there was no evidence of contamination in the media. Exudates (3 mL) were placed in 50 mL tubes, frozen then lyophilized. Dried exudates were resuspended in 1 mL LC-MS grade methanol (Honeywell Burdick & Jackson, Morristown, NJ, USA), vortexed for 10 sec, sonicated for 10 min then centrifuged for 5 min at 3200×g. The supernatant was collected in 1.5 mL microcentrifuge tubes then dried down with a Savant SpeedVac SPD111V (Thermo Scientific, Waltham, WA, USA) for 1 h with a final resuspension in ice-cold methanol containing 13C-15N amino acid internal standards (200 μL) and filtration through 0.22 μm centrifugal membranes (Nanosep MF, Pall Corporation, Port Washington, NY, USA) by centrifuging at 10,000×g for 5 min. Samples (50 μL) were transferred to LC-MS vials for metabolomics analysis, using hydrophilic interaction liquid chromatography for metabolite separations and electrospray ionization tandem mass spectrometry for metabolite detection. Chromatographic separations were performed with a SeQuant ZIC-HILIC 200 Å, 5 μm, 150×2.1 mm column (1.50454.0001, MilliporeSigma, Burlington, MA, USA) on an Agilent 1290 series UHPLC as previously described (32). MS1 and MS2 data were collected as previously described (32); briefly, eluted metabolites were detected using a Thermo Q Exactive hybrid Quadrupole-Orbitrap Mass Spectrometer equipped with a heated electrospray ionization source probe (Thermo Scientific, San Jose, CA, USA) using data dependent MS2 Top2 function with an m/z range of 70-1050 and stepped collision energies of 10, 20 and 30. Metabolomics data were analyzed using Metabolite Atlas with in-house Python scripts to obtain extracted ion chromatograms and peak heights for each metabolite (33); python code is available at https://github.com/biorack/metatlas. Metabolite identifications were verified by comparison with authentic chemical standards and validated based on three metrics: accurate mass (<5 ppm for positive ion mode, <20 ppm for negative ion mode), retention time within 0.5 min of prediction, and MS/MS fragmentation spectra similarity. Data from internal standards and quality control samples (included throughout the run) were analyzed to ensure consistent peak heights and retention times. Raw data files have been deposited in the JGI Genome Portal and are available for download here: https://genome.jgi.doe.gov/portal/201Tratabolomics_FD/201Tratabolomics_FD.info.html under project ID #1266724. Statistical comparisons (ANOVA and Tukey HSD, alpha=0.05) and heatmaps (peak heights scaled across samples relative to max for each compound; rows and columns clustered using complete clustering method with Euclidean distance measure) were generating in R using multcomp (34) and pheatmap (35) packages respectively. DNA extraction,

16S rRNA copy determination, library preparation and sequencing. The DNA from 0.25 g of soil from each fraction was extracted using the Qiagen® Power Soil DNA extraction kit. After fluorometric determination of DNA concentration in the extract (Qubit, Life Technologies, New York, USA), we used qPCR (quantitative real-time PCR) with a universal 16S rRNA gene primer set to determine 16S rRNA gene number as previously described (36). The V3 to V4 variable region of the 16S rRNA gene was targeted for amplification and Illumina sequencing as previously described (37), and run commercially. Raw sequence data were submitted to NCBI and are publicly available under BioProject ID PRJNA594377.

Bioinformatic analyses, phylogenetic assignments. The raw FASTQ file was demultiplexed within the MiSeq Illumina workflow under default parameters. Paired sequences were demultiplexed and analyzed via Qiime 2.10 (38), using the DADA2 plugin (39) to create a feature table with representative sequences (features) and their frequency of occurrence. For downstream diversity and composition analyses, the pipeline previously described (40) was followed, using Greengenes 13.8 release as a database. Community differences were assessed via permutational multivariate analysis of variance (PERMANOVA), performed on Bray-Curtis distance matrices of relative abundances derived from sequencing and used 9999 permutations. Microbial community differences were visualized using principal component analysis.

Metagenomics. DNA extracts from rhizosphere RAMS of AVPI and Col-0 plants (3 each) grown in artificial soil were obtained as described herein and sequenced independently. Paired-end raw reads were filtered with a quality score of 25 and assembled using MEGAHIT 1.2.9 (41). Scaffolds shorter 500 bp were discarded. Genes were predicted using Prodigal 2.6.3 (42). Unique genes were identified with CD-HIT 4.6.8 with an identity of 95% (43). The abundance of individual genes was estimated by re-mapping the clean reads to the unique genes with RSEM using Bowtie2 as an aligner (44). Gene functions were annotated with Megan Community version 6.0, with eggNOG, SEED, and Gene Ontology references (45). The abundance of genes was standardized against the total number of reads in each sample. The relative abundance of genes or groups of genes of interest were compared between the wild type and mutant RAMS using t test in R (46), with data logit transformed for proportion data. Ra Raw sequence data were submitted to NCBI and are publicly available under BioProject ID PRJNA646933

TABLE 1
PROPORTIONAL ABUNDANCE OF GENES IN QUERY AND CONTROL
PATHWAYS IN THE METAGENOME OF RHIZOSPHERE MICROBIOMES

| | | | | | | T-TEST | |
|---|---|---|---|---|---|---|---|
| | | | | | | (p-value)* | |
| | NAME | AVP1 | | COL-0 | | p < 0.1 | |
| | (enzyme | microbiome | | microbiome | | p < 0.05 | Percent |
| | gene | | Stand. | | Stand. | not | enrichment |
| TYPE | homologues) | Average | Dev. | Average | Dev. | significant | (AVPI/COL-0) |
| Query | Auxine | 1.6E−05 | 3.8E−06 | 1.5E−05 | 3.6E−06 | 0.17 | |
| | biosynthesis | | | | | | |
| | (Indoleacetamide | | | | | | |
| | hydrolase, | | | | | | |
| | Tryptophan 2- | | | | | | |
| | monooxygenase | | | | | | |
| Query | Homolactic | 2.3E−04 | 1.0E−05 | 1.7E−04 | 1.6E−06 | 2.E−04 | 41 |
| | fermentation | | | | | | |
| | (L-lactate | | | | | | |
| | dehydrogenase) | | | | | | |
| Query | Ethanol | 1.8E−03 | 7.8E−05 | 1.5E−03 | 5.1E−05 | 0.01 | 19 |
| | fermentation | | | | | | |
| | (Alcohol | | | | | | |
| | dehydrogenase, | | | | | | |
| | Pyruvate | | | | | | |
| | decarboxylase) | | | | | | |
| Query | Acetate | 5.6E−04 | 3.5E−05 | 4.6E−04 | 3.5E−05 | 0.02 | 22 |
| | fermentation | | | | | | |
| | (Pyruvate | | | | | | |
| | dehydrogenase, | | | | | | |
| | Pyruvate | | | | | | |
| | formate-lyase, | | | | | | |
| | Formate | | | | | | |
| | acetytransferase) | | | | | | |
| Query | Succinate | 2.3E−04 | 2.1E−05 | 1.8E−04 | 2.8E−05 | 0.07 | 30 |
| | fermentation | | | | | | |
| | (fumarate | | | | | | |
| | reductase) | | | | | | |
| Query | Butanediol | 2.0E−05 | 5.7E−07 | 1.3E−05 | 3.1E−06 | 0.03 | 56 |
| | fermentation | | | | | | |
| | (Acetolactate | | | | | | |
| | decarboxylase, | | | | | | |
| | Butanediol | | | | | | |
| | dehydrogenase) | | | | | | |
| Query | Fermentations | 1.8E−03 | 7.8E−05 | 1.5E−03 | 5.1E−05 | 5.E−03 | 19 |
| | (all above) | | | | | | |
| | (all | | | | | | |
| | fermentation | | | | | | |
| | enzyme | | | | | | |
| | homologs | | | | | | |
| | above) | | | | | | |
| Query | Nitrate/nitrite | 6.7E+03 | 8.0E+02 | 7.4E+03 | 2.4E+03 | 4.E−03 | −9 |
| | uptake | | | | | | |
| | (Nitrate ABC | | | | | | |
| | transporter [4 | | | | | | |
| | subunits] | | | | | | |
| | nitrate/nitrite | | | | | | |
| | transporter) | | | | | | |
| Query | Ammonium | 4.8E+03 | 1.1E+03 | 4.0E+03 | 1.8E+03 | 0.02 | 20 |
| | uptake | | | | | | |
| | (ammonium | | | | | | |
| | transporter) | | | | | | |
| Query | Phosphorous | 4.3E−04 | 1.8E−05 | 3.7E−04 | 1.9E−05 | 0.01 | 18 |
| | uptake | | | | | | |
| | (low-affinity | | | | | | |
| | inorganic P04 | | | | | | |
| | transporter, | | | | | | |
| | P04 ABC | | | | | | |
| | transporter [4 | | | | | | |
| | subunits], | | | | | | |
| | Na-dependent | | | | | | |
| | P04 | | | | | | |
| | transporter, | | | | | | |
| | phosphonate | | | | | | |
| | ABD | | | | | | |
| | transporter [4 | | | | | | |
| | subunits]) | | | | | | |
| Control | Cell division | 1.5E−04 | 4.8E−06 | 1.5E−04 | 9.8E−06 | 0.91 | |
| | (FtsA, FStZ) | | | | | | |
| Control | Glycolisis | 1.3E−04 | 2.6E−05 | 1.2E−04 | 2.9E−05 | 0.65 | |
| | (Fructose- | | | | | | |
| | bisphosphate | | | | | | |
| | aldolase, | | | | | | |
| | Triose | | | | | | |
| | phosphate | | | | | | |
| | isomerase) | | | | | | |
| Control | DNA | 4.4E−04 | 2.7E−05 | 3.9E−04 | 5.2E−05 | 0.21 | |
| | replication | | | | | | |
| | (Replicative | | | | | | |
| | DNA helicase, | | | | | | |
| | DNA | | | | | | |
| | polymerase III | | | | | | |
| | [8 subunits]) | | | | | | |
| Control | Fatty acid | 1.0E−05 | 3.9E−06 | 1.6E−05 | 1.1E−06 | 0.12 | |
| | biosynthesis | | | | | | |
| | (3-oxoacy-ACP | | | | | | |
| | synthase, | | | | | | |
| | Acyl carrier | | | | | | |
| | protein ACP) | | | | | | |

*Data logit transformed to account for use of proportional data

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1. A method of increasing plant growth, increasing plant yield, or improving soil quality, the method comprising cultivating in soil a genetically engineered plant that produces an enriched root associated microbiome (RAM), wherein the enriched RAM improves the soil quality, increases the plant growth of one or more co-cultivated plants, and/or increases the plant yield of one or more co-cultivated plants.

2. (canceled)

3. The method of claim 1, wherein increased plant growth, increased plant yield, or improved soil quality is indicated by increased root yield, increased shoot yield, or increased crop yield.

4. The method of claim 1, wherein plant yield comprises plant foliage and/or crop yield.

5. The method of claim 1, wherein the genetically engineered plant is engineered to over express a type 1 H+-pyrophosphatase.

6. The method of claim 1, wherein the genetically engineered plant is of the genus Arabidopsis.

7. (canceled)

8. The method of claim 1, wherein the one or more co-cultivated plants are selected from the group consisting of one or more a wildtype plants, one or more crop plants, or a combination thereof.

9. (canceled)

10. The method of claim 8, wherein the one or more crop plants are selected from the group consisting of rice, barley, tobacco, cotton, alfalfa, maize, wheat, or a combination thereof.

11. The method of claim 1, wherein the enriched RAM comprises one or more viable microorganisms selected from the group consisting of bacteria, fungi, yeast, unicellular algae, viruses, phages, or a combination thereof.

12-13. (canceled)

14. The method of claim 11, wherein the one or more viable microorganisms comprise enriched fermentation capacity.

15. (canceled)

16. A method of increasing plant growth, increasing plant yield, or improving soil quality, the method comprising applying to the soil of a plant an agricultural composition comprising an enriched root associated microbiome (RAM) or a portion thereof.

17. (canceled)

18. The method of claim 16, wherein the agricultural composition comprising an enriched root associated microbiome (RAM) is provided in an amount effective to increase plant growth, increase plant root yield, increase leaf foliage, increase plant shoot yield, increase crop yield, and/or improve soil quality.

19. The method of claim 16, wherein the enriched RAM comprises one or more viable microorganisms selected from the group consisting of bacteria, fungi, yeast, unicellular algae, viruses, phages, or a combination thereof.

20-21. (canceled)

22. The method of claim 19, wherein the one or more viable microorganisms comprise enriched fermentation capacity.

23. (canceled)

24. The method of claim 16, wherein the enriched root associated microbiome (RAM) is obtained by cultivating a genetically engineered plant in a soil composition for a period of time, thereby providing a composition comprising an enriched RAM, and obtaining therefrom the composition comprising the enriched RAM.

25. An agricultural composition comprising an enriched root associated microbiome (RAM) or a portion thereof, wherein the enriched RAM is obtained from a genetically engineered plant.

26. The agricultural composition of claim 25, wherein the genetically engineered plant is modified to overexpress a type 1 H+-pyrophosphatase.

27. The agricultural composition of claim 25, wherein the genetically engineered plant is of the genus Arabidopsis.

28. (canceled)

29. The agricultural composition of claim 25, wherein the enriched RAM comprises one or more viable microorganisms selected from the group consisting of bacteria, fungi, yeast, unicellular algae, viruses, phages, or a combination thereof.

30-31. (canceled)

32. The agricultural composition of claim 29, wherein the one or more viable microorganisms comprise enriched fermentation capacity.

33. (canceled)

34. The agricultural composition of claim 25, wherein the enriched RAM further comprises metabolites of nitrogen metabolism, 2,3-butanediol, at least one other plant nutrient, or a combination thereof.

35-36. (canceled)
